(19) United States
(12) Patent Application Publication
(10) Pub. No.: US 2008/0287477 A1
(43) Pub. Date: Nov. 20, 2008

(54) Novel Compounds as Modulators of Ppar

(75) Inventors: James W. Malecha, San Diego, CA (US); Andrew K. Lindstrom, Encinitas, CA (US); Sergio G. Duron, San Diego, CA (US); Steven P. Govek, San Diego, CA (US)

Correspondence Address: GLOBAL PATENT GROUP - KAL;ATTN: MS LAVERN HALL, 10411 Clayton Road, Suite 304, St. Louis, MO, 63131, US

(73) Assignee: KALYPSYS, INC., SAN DIEGO, CA (US)

(21) Appl. No.: 12/083,426

(22) PCT Filed: Oct. 11, 2006
(86) PCT No.: PCT/US2006/400791

§ 371 Date: Apr. 24, 2008

(51) Int. Cl. A61K 31/192; C07D 239/42; C07C 311/29; A61P 29/00; A61P 17/00; A61P 27/02; A61P 19/00; A61K 31/505; A61P 3/00

(52) U.S. Cl. 514/275; 544/332; 562/426; 514/562

(57) ABSTRACT

Compounds as modulators of peroxisome proliferator activated receptors, pharmaceutical compositions comprising the same, and methods of treating disease using the same are disclosed.

### CROSS REFERENCE TO RELATED APPLICATION

This application claims the benefit of priority of U.S. provisional application Ser. No. 60/726,402, filed on Oct. 12, 2005, the disclosure of which is hereby incorporated by reference as if written herein in its entirely.

### FIELD OF THE INVENTION

The present invention relates to novel sulfonyl-substituted bicyclic aryl derivatives and methods for treating various diseases by modulation of nuclear receptor mediated processes using these compounds, and in particular processes mediated by peroxisome proliferator activated receptors (PPARs).

### BACKGROUND OF THE INVENTION

Peroxisome proliferators are a structurally diverse group of compounds which, when administered to mammals, elicit dramatic increases in the size and number of hepatic and renal peroxisomes, as well as concomitant increases in the capacity of peroxisomes to metabolize ratty acids via increased expression of the enzymes required for the β-oxidation cycle (Lazarow and Fujiki, Ann. Rev. Cell Biol. 1:489-530 (1935): Vamecq and Draye, Essays Biochem. 24:1115-225 (1989); and Nelail et al. Cancer Res. 48:5316-5324 (1988)). Compounds that activate or otherwise interact with one or more of the PPARs have been implicated in the regulation of triglyceride and cholesterol levels in animal models. Compounds included in this group are the fibrate class of hypolipidemic drugs, herbicides, and phthalate plasticizers (Reddy and Lalwani, Crit. Rev. Toxicol. 12:1-58 (1983)). Peroxisome proliferation can also be elicited by dietary or physiological factors such as a high-fat diet and cold acclimatization.

Biological processes modulated by PPAR are those modulated by receptors, or receptor combinations, which are responsive to the PPAR receptor Uganda. These processes include, for example, plasma lipid transport and fatty acid catabolism, regulation of insulin sensitivity and blood glucose levels, which are involved in hypoglycemia/hyperinsulinemia (resulting from, for example, abnormal pancreatic beta cell function, insulin secreting tumors end/or autoimmune hypoglycemia due to autoantibodies to insulin, the insulin receptor, or autoantibodies that are stimulatory to pancreatic beta cells), macrophage differentiation which lead to the formation of atherosclerotic plaques, inflammatory response, carcinogenesis, hyperplasia, and adipocyte differentiation.

Subtypes of PPAR include PPAR-alpha, PPAR-delta (also known as NUCl, PPAR-beta and FAAR) and two isoforms of PPAR-gamma. These PPARs can regulate expression of target genes by binding to DNA sequence elements, termed PPAR response elements (PPRE). To date, PPRE's have been identified in the enhancers of a number of genes encoding proteins that regulate lipid metabolism suggesting that PPARs play a pivotal role in the adipogenic signaling cascade and lipid homeostasis (H. Keller and W. Wahli, Trends Endoodn. Met. 291-296, 4 (1993)).

Insight into the mechanism whereby peroxisome proliferators exert their pleiotropic effects was provided by the identification of a member of the nuclear hormone receptor superfamily activated by these chemicals (Isseman and Green, Nature 347-645-650 (1990)). The receptor, termed PPAR-alpha (or alternatively, PPARα), was subsequently shown to be activated by a variety of medium and long-chain fatty acids and to stimulate expression of the genes encoding rat acyl-CoA oxidase and hydrolase-dehydrogenase (enzymes required for peroxisomal β-oxidation), as well as rabbit cytochrome P450 4A6, a fatty acid ω-hydroxylase (Gottlicher et al., Proc. Natl. Acad. Sci. USA 89:4653-4657 (1992); Tugwood et al., EMBO J 11:433-439 (1992); Bardot et al., Biochem. Biophys. Res. Comm. 192:37-45 (1993); Muerhoff et al. Biol. Chem. 267: 19051-19053 (1992); and Marcus et al., Proc. Natl. Acad. Sci. USA 90(12): 5723-5727 (1993).

Activators of the nuclear receptor PPAR-gamma (or alternatively, PPARγ), for example troglitazone, have been clinically shown to enhance insulin-action, to reduce serum glucose and to have small but significant effects on reducing serum triglyceride levels in patients with Type 2 diabetes. See, for example, D. E. Kelly et al., Curr. Opin. Endocrinol. Diabetes, 90-96, 5 (2), (1998); M. D. Johnson et al., Ann. Pharmacother., 337-348, 32 (3), (1997); and M. Leutenegger et al., Curr. Ther. Res., 403-416, 58(7), (1997).

PPAR-delta (or alternatively, PPARδ) initially received much less attention than the other PPARs because of its ubiquitous expression and the unavailability of selective ligands. However, genetic studies and recently developed synthetic PPAR-δ agonists have helped reveal its role as a powerful regulator of fatty acid catabolism and energy homeostasis. Studies in adipose tissue and muscle have begun to uncover the metabolic functions of PPAR-δ. Transgenic expression of an activated form of PPAR-δ in adipose tissue produces lean mice that are resistant to obesity, hyperlipidemia and tissue steatosis induced genetically or by a high-fat diet. The activated receptor induces genes required for fatty acid catabolism and adaptive thermogenesis. Interestingly, the transcription of PPAR-γ target genes for lipid storage and lipogenesis remain unchanged. In parallel, PPAR-δ-deficient mice challenged with a high-fat diet show reduced energy uncoupling and are prone to obesity. Together, these data identify PPAR-δ as a key regulator of fat-burning, a role that opposes the fat-storing function of PPAR-γ. Thus, despite their close evolutionary and structural kinship, PPAR-γ and PPAR-δ regulate distinct genetic networks. In skeletal muscle, PPAR-δ likewise upregulates fatty oxidation and energy expenditure, to a far greater extent than does the lesser-expressed PPAR-α. (Evans R M et al 2004 Nature Med 1-7, 10(4), 2004)

PPAR-δ is broadly expressed in the body and has been shown to be a valuable molecular target for treatment of dyslipidemia and other diseases. For example, in a recent study in insulin-resistant obese rhesus monkeys, a potent and selective PPAR-delta compound was shown to decrease VLDL and increase HDL in a dose response manner (Oliver et el., Proc. Natl. Acad. Sci. U.S.A. 98:5305, 2001).

Because there are three isoforms of PPAR and all of them have been shown to play important roles in energy homeostasis and other important biological processes in human body and have been shown to be important molecular targets for treatment of metabolic and other diseases (see Willson, et al. J. Med. Chem. 43:527-550 (2000)), it is desired in the art to identify compounds which are capable of interacting with multiple PPAR isoforms or compounds which are capable of selectively interacting with only one of the PPAR isoforms, preferably PPARδ. Such compounds would find a wide variety of uses, such as, for example, in the treatment or prevention of obesity, for the treatment or prevention of diabetes, dyslipidemia, metabolic syndrome X and other uses.

Several PPAR-modulating drugs have been approved for use in humans. Fenofibrate and gemfibrozil are PPARα modulators: pioglitazone (Actos, Takeda Pharmaceuticals and Eli Lilly) and rosiglitazone (Avandia, GlaxcoSmithKline) are PPARγ modulators. However, oil of these compounds have liabilities us potential carcinogens, having been demonstrated to have proliferative effects leading to cancers of various types (colon; bladder with PPARα modulators and liver with PPARγ modulators) in rodent studies. Therefore, a need exists to identify other modulators of PPARs which lack these liabilities. Selective modulators of PPARδ may provide an opportunity for such improvements, and may even prove useful in the treatment of cancers, including colon, skin, and lung cancers.

### SUMMARY OF THE INVENTION

The present invention provides a class of compounds useful as modulators of PPAR, having structural Formula (I)

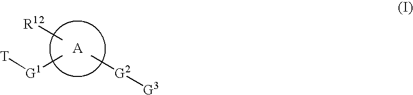

wherein:

A is selected from the group consisting of aryl, heteroaryl, cycloalkyl, and heterocycloalkyl, any of which may be optionally substituted;

R¹² is selected from the group consisting of hydrogen, lower alkyl, lower alkenyl, lower heteroalkyl, and lower alkoxy; R¹² may join together with a carbon atom in G¹ to form a five to eight-membered carbocycle or heterocycle, having structural Formula (II):

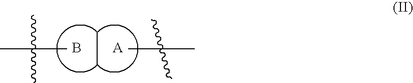

B is a saturated, partially saturated, or unsaturated hydrocarbon chain, optionally containing one or more heteroatoms, to form an optionally substituted five- to eight-membered carbocycle or heterocycle;

T is selected from the group consisting of —C(O)OH, —C(O)NH₂, and tetrazole;

G¹ is selected from the group consisting of —(CR¹R²)ₙ—, -Z(CR¹R²⁾ᵣ—, —(CR¹R²)ᵣZ—, —(CR¹R²)ᵣZ(CR¹R²)ₛ—;

Z is O, S, or NR⁶;

n is 1 to 4;

r and s are 0 to 2;

R¹ and R² are each independently selected from the group consisting of hydrogen, halogen, lower alkyl, lower alkoxy, and lower perhaloalkyl, or R¹ and R² together may form a cycloalkyl;

G² is —Y(CR³R⁴)ₚW(CR³R⁴)ₘ—;

Y is S, —SO₂N(R⁵)— or NR⁶;

W is O, S or —NR⁶;

p is 2 to 6:

m is 0, 1 or 2;

R³ and R⁴ are each independently selected from the group consisting of hydrogen, halogen, hydroxy, optionally substituted lower alkyl, optionally substituted lower alkoxy, optionally substituted heteroalkyl, optionally substituted cycloalkyl, lower perhaloalkyl, lower perhaloalkoxy, nitro, cyano, NH₂, and —C(O)OR¹¹, or R³ and R⁴ together may form a cycloalkyl;

R¹¹ is selected from the group consisting of hydrogen and optionally substituted lower alkyl;

R⁵ and R⁶ are each independently selected from the group consisting of hydrogen, optionally substituted lower alkyl, optionally substituted lower alkenyl, optionally substituted lower alkynyl, optionally substituted heteroalkyl, optionally substituted aryl, and optionally substituted heteroaryl;

G³ is selected from the group consisting of hydrogen, optionally substituted aryl, optionally substituted heteroaryl, optionally substituted cycloalkyl, optionally substituted cycloheteroalkyl, and —N═C(R⁷R⁸);

R⁷ and R⁸ are each individually selected from the group consisting of hydrogen, optionally substituted aryl, optionally substituted heteroaryl, optionally substituted cycloalkyl, and optionally substituted cycloheteroalkyl; and

wherein said effect is selected from the group consisting of modulation of PPARδ, upregulation of expression of GLUT4 in adipose tissue, reduction of expression of NPC1L1, raising of HDL, lowering of LDLc, shifting of LDL particle size from small dense to normal LDL, inhibition of cholesterol absorption, reduction of triglycerides, decrease of insulin resistance, lowering of blood pressure, promotion of wound healing, reduction of scarring, and treatment of a PPARδ-mediated disease.

In preferred embodiments, the compounds of the invention are selective modulators of PPARδ.

In other aspects, the present invention provides methods of, alone or in combination, raising HDL, lowering LDLc, shifting LDL particle size from small dense to normal LDL, and inhibiting cholesterol absorption, comprising the administration of a therapeutic amount of a compound of the invention.

In other aspects, the present invention provides methods for treating metabolic disorders and related conditions, in a human or animal subject in need of such treatment comprising administering to said subject an amount of a compound of formula (I) effective to reduce or prevent said disorders or conditions in the patient.

In other aspects, the invention provides for pharmaceutical compositions comprising the compounds of the invention, together with one or more pharmaceutically acceptable diluents or carriers. In related aspects, the invention provides for pharmaceutical compositions comprising the compounds of the invention and one or more additional agents, for the treatment of metabolic disorders.

### DETAILED DESCRIPTION OF THE INVENTION

In certain embodiments, the compounds of the present invention have structural Formula (I) wherein:

T is —CO(O)H;

G¹ is —(CR¹R²)ₙ—;

R¹ and R² are each independently selected from the group consisting of hydrogen, halogen, lower alkyl, lower alkoxy, and lower perhaloalkyl;

G² is —Y(CR³R⁴)ₚW(CR³R⁴)ₘ—;

W is O, or —NR⁶;

p is 2; and

G³ is selected from the group consisting of optionally substituted aryl, optionally substituted heteroaryl, optionally substituted cycloalkyl, optionally substituted cycloheteroalkyl, optionally substituted cycloalkenyl, and —N═C(R⁷R⁸).

In further embodiments, the compounds of the present invention

wherein:

R³ and R⁴ are each hydrogen;

and Y is —SO₂N(R⁵)—.

In yet further embodiments, the compounds of the present invention

wherein:

A is optionally substituted phenyl;

R¹² is hydrogen; and

R¹ and R² are each independently selected from the group consisting of hydrogen, methyl, ethyl, and propyl;

In yet further embodiments, the compounds of the invention wherein W is —NR⁶.

In yet further embodiments, the compounds of the invention wherein m is 1.

In yet further embodiments, the compounds of the invention wherein G³ is optionally substituted aryl.

In yet further embodiments, the compounds of the invention wherein said aryl is optionally substituted with one or more of the following: halogen, perhaloalkyl, and perhaloalkoxy.

In yet further embodiments, the compounds of the invention wherein said aryl is substituted with perhaloalkoxy.

In yet further embodiments, the compounds of the invention wherein said perhaloalkoxy is trifluoromethoxy.

In yet further embodiments, the compounds of the invention wherein said trifluoromethoxy substitutes said aryl in the para position.

In certain embodiments, the compounds of the invention

wherein:

W is O;

m is 0; and

G₃ is optionally substituted aryl.

In certain embodiments, the compounds of the invention

wherein:

W is O;

m is 0; and

G₃ is optionally substituted heteroaryl.

In certain embodiments, the compounds of the invention

wherein:

W is O;

m is 0; and

G₃ is —N═C(R⁷R⁸).

In further embodiments, the compounds of the invention wherein at least one R⁷ and R⁸ is optionally substituted aryl.

The yet further embodiments, the compounds of the invention wherein both R⁷ and R⁸ is optionally substituted aryl.

In preferred embodiments, the compounds of the present invention

wherein:

A is optionally substituted phenyl;

R¹² joins together with a carbon atom in G¹ to form a five to eight-membered carbocycle or heterocycle; and

R¹ and R² are each independently selected from the group consisting of hydrogen, methyl, ethyl, and propyl.

In yet more preferred embodiments, the compounds of the present invention having a structural Formula (III) or (IV) as follows:

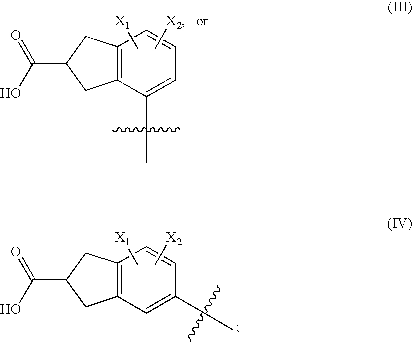

wherein X¹ and X² are each independently selected from the group consisting of hydrogen, halogen, hydroxy, optionally substituted lower alkyl, optionally substituted cycloalkyl, optionally substituted heteroalkyl, optionally substituted cycloheteroalkyl, optionally substituted lower alkynyl, perhaloalkyl, perhaloalkoxy, optionally substituted lower alkoxy, nitro, cyano, and NH₂.

In yet more preferred embodiments, the compounds of the present invention

wherein:

W is O;

m is 0;

X₁ and X₂ are each hydrogen; and

G₃ is optionally substituted aryl.

In yet more preferred embodiments, the compounds of the present invention

wherein:

W is O:

m is 0;

X₁ and X₃ are each hydrogen; and

G₃ is optionally substituted heteroaryl.

In yet more preferred embodiments, the compounds of the present invention

wherein:

W is O;

m is 0;

X₁ and X₂ are each hydrogen; and

G₃ is —N═C(R⁷R⁸).

In yet more preferred embodiments, the compounds of the present invention wherein at least one of R⁷ and R⁸ is optionally substituted aryl.

In yet more preferred embodiments, the compounds of the present invention wherein both R⁷ and R⁸ are optionally substituted aryl.

In yet more preferred embodiments, the compounds of the present invention

wherein:

W is N;

m is 1;

X₁ and X₃ are each hydrogen;

R³ and R⁴ are hydrogen; and

G³ is optionally substituted aryl.

In certain embodiments, the compounds of the present invention having a structural Formula (V) as follows:

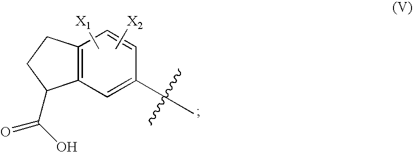

wherein X¹ and X² are each independently selected from the group consisting of hydrogen, halogen, hydroxy, optionally substituted lower alkyl, optionally substituted cycloalkyl, optionally substituted heteroalkyl, optionally substituted cycloheteroalkyl, optionally substituted lower alkynyl, perhaloalkyl, perhaloalkoxy, optionally substituted lower alkoxy, nitro, cyano, and NH₃.

In yet further embodiments, the compounds of the present invention

wherein:

W is O;

m is 0,

X₁ and X₂ are each hydrogen; and

G₃ is optionally substituted aryl.

In certain embodiments, the compounds of the present invention having a structural Formula (VI) as follows:

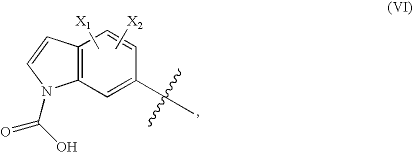

The present invention discloses that novel compounds disclosed herein can modulate at least one peroxisome proliferator-activated receptor (PPAR) function. Compounds described herein may be activating both PPAR-delta and PPAR-gamma or PPAR-alpha and PPAR-delta, or all three PPAR subtypes, or selectively activating predominantly PPAR-gamma, PPAR-alpha or PPAR-delta.

The present invention discloses a method of modulating at least one peroxisome proliferator-activated receptor (PPAR) function comprising the step of contacting the PPAR with a compound of Formula I, as described herein. The change in cell phenotype, cell proliferation, activity of the PPAR, expression of the PPAR or binding of the PPAR with a natural binding partner may be monitored. Such methods may be modes of treatment of disease, biological assays, cellular assays, biochemical assays, or the like.

The present invention describes methods of treating a PPAR-mediated disease or metabolic disorder comprising identifying a patient having said disease, and administering a therapeutically effective amount of a compound of Formula I, as described herein, to a patient. Thus, in certain embodiments, the disease to be treated by the methods of the present invention is selected from the group consisting of obesity, diabetes, hyperinsulinemia, metabolic syndrome X, polycystic ovary syndrome, climacteric, disorders associated with oxidative stress, inflammatory response to tissue injury, pathogenesis of emphysema, ischemia-associated organ injury, doxorubicin-induced cardiac injury, drug-induced hepatotoxicity, atherosclerosis, and hypertoxic lung injury. In another aspect, the present invention relates to a method of modulating at least one peroxisome proliferator-activated receptor (PPAR) function comprising the step of contacting the PPAR with a compound of Formula I, as described herein. The change in cell phenotype, cell proliferation, activity of the PPAR, or binding of the PPAR with a natural binding partner may be monitored. Such methods may be modes of treatment of disease, biological assays, cellular assays, biochemical assays, or the like. In certain embodiments, the PPAR may be selected from the group consisting of PPARα, PPARδ, and PPARγ. In preferred embodiments, the PPAR is PPARδ.

The invention also discloses the use of a PPAR-delta modulator compound according to the invention for the manufacture of a medicament for raising HDL, lowering LDLc, shifting LDL particle size from small dense to normal LDL, or Inhibiting cholesterol absorption.

The invention discloses methods of treatment of a PPAR-delta mediated disease or condition comprising administering a therapeutically effective amount of a compound according the present invention or a pharmaceutically acceptable salt, ester, amide, or prodrug thereof. In certain embodiments, the present invention discloses: methods for treating Type 2 diabetes, decreasing insulin resistance or lowering blood pressure in a subject; methods for treating atherosclerotic diseases including vascular disease, coronary heart disease, cerebrovascular disease and peripheral vessel disease in a subject; methods for treating cancers including colon, skin, and lung cancers in a subject; and methods for treating inflammatory diseases, including rheumatoid arthritis, asthma, osteoarthritis and autoimmune disease in a subject, all comprising the administration of a therapeutic amount of a PPAR-delta modulator compound according to the present invention.

The invention further discloses compounds of the invention or pharmaceutical compositions thereof for use in the manufacture of a medicament for the prevention or treatment of a disease or condition ameliorated by the modulation of a PPAR-delta. Certain embodiments of the invention include the use of a PPAR-delta modulator compound having structural formula (I) for the manufacture of a medicament for the treatment of: Type 2 diabetes, or for decreasing insulin resistance or lowering blood pressure; atherosclerotic diseases including vascular disease, coronary heart disease), cerebrovascular disease and peripheral vessel disease; cancers including colon, skin, and lung cancers; and inflammatory diseases, including rheumatoid arthritis, asthma, osteoarthritis and autoimmune disease, in a patient in need thereof.

Another aspect of the invention are compounds of the invention or pharmaceutical compositions thereof for use in the treatment of disease or condition ameliorated by the modulation of a PPAR-delta wherein said PPAR-delta mediated disease or condition is dyslipidemia, metabolic syndrome X, heart failure, hypercholesteremia, cardiovascular disease, type II diabetes mellitus, type I diabetes, insulin resistance hyperlipidemia, obesity, anorexia bulimia, inflammation and anorexia nervosa.

Another aspect of the invention are compounds, pharmaceutically acceptable prodrugs, pharmaceutically active metabolites, or pharmaceutically acceptable salts thereof and having an EC₅₀ value less than 5 μM as measured by a functional cell assay.

Another aspect of the invention are methods of modulating a peroxisome proliferator-activated receptor (PPAR) function comprising contacting said PPAR with a compound of the present invention and monitoring a change in cell phenotype, cell proliferation, activity of said PPAR, or binding of said PPAR with a natural binding partner.

Another aspect of the invention are method of modulating a peroxisome proliferator-activated receptor (PPAR) wherein the PPAR is selected from the group consisting of PPAR-alpha, PPAR-delta, and PPAR-gamma.

Another aspect of the invention are methods of treating a disease comprising identifying a patient in need thereof, and administering a therapeutically effective amount of a compound of the present invention 10 said patient wherein said disease is selected from the group consisting of obesity, diabetes, hyperinsulinemia, metabolic syndrome X, polycystic ovary syndrome, climacteric, disorders associated with oxidative stress, inflammatory response to tissue injury, pathogenesis of emphysema, ischemia-associated organ injury, doxorubicin-induced cardiac Injury, drug-induced hepatotoxicity, atherosclerosis, and hypertoxic lung injury.

Another aspect of the invention are compounds which modulates a peroxisome proliferator-activated receptor (PPAR) function, preferably wherein said PPAR is selected from the group consisting of PPARα, PPARδ, and PPARγ.

Another aspect of the invention are compounds or composition for use in the treatment of a disease or condition ameliorated by the modulation of a PPAR such as PPARα, PPARδ, and PPARγ, wherein the disease or condition is dyslipidemia, metabolic syndrome X, heart failure, hypercholesteremia, cardiovascular disease, type II diabetes mellitus, type I diabetes, insulin resistance hyperlipidemia, obesity, anorexia bulimia, inflammation and anorexia nervosa.

Another aspect of the invention are compounds or compositions according for use in the manufacture of a medicament for the prevention or treatment of disease or condition ameliorated by the modulation of a PPAR such as PPARα, PPARδ, and PPARγ.

As used in the present specification the following terms have the meanings indicated:

The term “acyl,” as used herein, alone or in combination, refers to a carbonyl attached to on alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20, preferably 2 to 6, carbon atoms. Alkenylene refers to a carbon-carbon double bond system attached at two or more positions such as ethenylene [(—CH═CH—), (—C::C—)]. Examples of suitable alkenyl radicals include ethenyl, propenyl, 2-methylpropenyl, 1,4-butadienyl and the like.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to and including 20, preferably 1 to 10, and more preferably 1 to 6, carbon atoms. Alkyl groups may be optionally substituted as defined herein. Examples of alkyl radicals include methyl, ethyl, n-propyl, isopropyl, n-butyl, isobutyl, sec-butyl, ten-butyl, pentyl, iso-amyl, hexyl, octyl, noyl end the like. The term “alkylene,” as used herein, alone or in combination, refers to a saturated aliphatic group derived from a straight or branched chain saturated hydrocarbon attached at two or more positions, such as methylene (—CH₂—).

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl.” as used herein, alone or in combination, refers to a straight-chain or branched chain hydrocarbon radical having one or more triple bonds and containing from 2 to 20, preferably from 2 to 6, more preferably from 2 to 4, carbon atoms. “Alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the patent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄═ derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group—with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is 09 defined herein.

The term “cyano,” as used herein, alone or in combination, refers to —CN.

The term “cycloalkyl,” as used herein, alone or in combination, refers to a saturated or partially saturated monocyclic, bicyclic or tricyclic alkyl radical wherein each cyclic moiety contains from 3 to 12, preferably five to seven, carbon atom ring members and which may optionally be a benzo fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydronaphthalene, octahydronapthalene as well as the multicyclic (multicentered) saturated or partially unsaturated type. The latter type of isomer is exemplified in general by, bicyclo[1,1,1]pentane, camphor, adamantane, and bicyclo[3.2.1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein die nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to 3 to 7 membered, preferably 5 to 7 membered, unsaturated heteromonocyclic rings, or fused polycyclic rings in which at least one of the fused rings is unsaturated, wherein at least one atom is selected from the group consisting of O, S, and N. The term also embraces fused polyoyclic groups wherein heterocyclic radicals are fused with aryl radicals, wherein heteroaryl radicals are fused with other heteroaryl radicals, or wherein heteroaryl radicals are fused with cycloalkyl radicals. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, Isothiazolyl, indolyl, isoindolyl, indolizinyl, benzimidazolyl, quinolyl, isoquinolyl, indazolyl, benzotriazolyl, benzoxazolyl, benzoxadiazolyl, benzothiazolyl, benzothiadiazolyl, benzofuryl, benzothienyl, tetrazolopyridazinyl, thienopyridine, furopyridine, pyrrolopyridine end the like.

The terms “heterocycloalkyl” and, interchangeably, “heterocycle,” as used herein, alone or in combination, each refer to a saturated, partially unsaturated, or fully unsaturated monocyclic, tricyclic, or tricyclic heterocyclic radical containing at least one, preferably 1 to 4, and more preferably 1 to 2 heteroatoms as ring members, wherein each said heteroatom may be independently selected from the group consisting of nitrogen, oxygen, and sulfur, and wherein there are preferably 3 to 8 ring members in each ring, more preferably 3 to 7 ring members in each ring, and most preferably 5 to 6 ring members in each ring. “Heterocycloalkyl” and “heterocycle” are intended to include sulfates, sulfoxides, N-oxides of tertiary nitrogen ring members, and carbocyclic fused and benzo fused ring systems: additionally, both terms also include systems where a heterocycle ring is fused to an aryl group, as defined herein, or on additional heterocycle group. Heterocycle groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The terms “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of this invention.

The term. “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to en alkyl group where all of the hydrogen atoms are replaced by halogen atoms.

The terms “sulfonate,” “sulfonic acid,” and “sulfonio,” as used herein, alone or in combination, refer the —SO₃H group and its onion as the sulfonic acid is used in salt formation.

The term “sulfanyl,” as used herein, alone or in combination, refers to —S—.

The term “sulfinyl,” as used herein, alone or in combination, refers to —S(O)—.

The terra “sulfonyl,” as used herein, alone or in combination, refers to —SO₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “this” and “thio,” as used herein, alone or in combination, refer to a —S— group or on ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomelhanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethylsilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfanyl, arylsulfinyl, arylsulfonyl, arylthio, sulfonate, sulfonic acid, trisubstituted silyl N₃, SH, SCH₂, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds of the present invention can exist in unsolvated as well as solvated forms with pharmaceutically acceptable solvents such as water, ethanol, and the like. In genera), the solvated forms are considered equivalent to the unsolvated forms for the purposes of the present invention.

The term “bond” refers to a covalent linkage between two atoms, or two moieties when the atoms joined by the bond are considered to be part of larger substructure. A bond may be single, double, or triple unless otherwise specified.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“PPAR modulator” is used herein to refer to a compound that exhibits an IC₅₀ with respect to PPAR activity of no more than about 100 μM and more typically not more than about 50 .mu.M, as measured in the PPAR assay described generally hereinbelow. “IC₅₀” is that concentration of inhibitor which reduces the activity of an enzyme (e.g., PPAR) to half-maximal level. Representative compounds of the present invention have been discovered to exhibit inhibitory activity against PPAR. Compounds of the present invention preferably exhibit an IC₅₀ with respect to PPAR of no more than about 10 μM, more preferably, no more than about 5 μM, even more preferably not more than about 1 μM, and most preferably, not more than about 200 nM, as measured in the PPAR assays described herein.

As used herein, reference to “treatment” of a patient is intended to include prophylaxis. The term “patient” means all mammals including humans. Examples of patients include humans, cows, dogs, cats, goats, sheep, pigs, and rabbits.

The term “therapeutically effective amount” as used herein refers to that amount of the compound being administered which will relieve to some extent one or more of the symptoms of the disease, condition or disorder being treated. In reference to the treatment of diabetes or dyslipidemia a therapeutically effective amount refers to that amount which has the effect of (1) reducing the blood glucose levels; (2) normalizing lipids, e.g. triglycerides, low-density lipoprotein; (3) relieving to some extent (or, preferably, eliminating) one or more symptoms associated with the disease, condition or disorder to be treated; and/or (4) raising HDL.

The terms “enhance” or “enhancing” means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system. When used in a patient, amounts effective for this use will depend on the severity and course of the disease, disorder or condition (including, but not limited to, metabolic disorders), previous therapy, the patient's health status and response to the drugs, and the judgment of the treating physician. It is considered well within the skill of the art for one to determine such enhancing-effective amounts by routine experimentation.

Unless otherwise indicated, when a substituent is deemed to be “optionally substituted,” it is meant that the substituent is a group that may be substituted with one or more group(s) individually and independently selected from alkyl, cycloalkyl, aryl, heteroaryl, heteroalicyclic, hydroxy, alkoxy, perhaloalkoxy, (preferably perfluoroalkyloxy), mono or dihaloalkoxy, aryloxy, mercapto, alkylthio, arylthio, cyano, halo, carbonyl, thiocarbonyl, O-carbamyl. N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, perhaloalkyl, perfluoroalkyl, silyl, trihalomethanesulfonyl, and amino, including mono-, and di-substituted amino groups, and the protected derivatives thereof. The protecting groups that may form the protective derivatives of the above substituents are known to those of skill in the art and may be found in references such as Greene and Wuts, above.

Molecular embodiments of the present invention may possess one or more chiral centers and each center may exist in the R or S configuration. The present invention includes all diastereomeric, enantiomeric, and epimeric forms as well as the appropriate mixtures thereof. Stereoisomers may be obtained, if desired, by methods known in the art as, for example, the separation of stereoisomers by chiral chromatographic columns. Additionally, the compounds of the present invention may exist as geometric isomers. The present invention includes all cis, trans, syn, anti, entgegen (E), and zusammen (Z) isomers as well as the appropriate mixtures thereof.

In some situations, compounds may exist as tautomers. All tautomers are included within Formula I and are provided by this invention.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

In another aspect, the present invention relates to a method of treating a disease comprising identifying a patient in need thereof, and administering a therapeutically effective amount of a compound of Formula I, as described herein, to the patient.

The third subtype of PPARs, PPARδ (PPARδ, NUCl), is broadly expressed in the body and has been shown to be a valuable molecular target for treatment of dyslipidemia and other diseases. For example, in a recent study in insulin-resistant obese rhesus monkeys, a potent and selective PPARδ compound was shown to decrease VLDL and increase HDL in a dose response manner (Oliver et al., Proc, Natl. Acad. Sci. U.S.A. 98:5305, 2001). Also, in a recent study in wild-type and HDL-lacking, ABCA1^{-/-} mice, a different potent and selective PPARδ compound was shown to reduce fractional cholesterol absorption in the intestine, and coincidentally reduce expression of the cholesterol-absorption protein NPC1L1 (van der Veen et al., 1. Lipid Res. 2005 46: 526-534).

The compounds of the invention are useful in the treatment of a disease or condition ameliorated by the modulation of an PPAR-delta. Specific diseases and conditions modulated by PPAR-delta and for which the compounds and compositions are useful include but are not limited to dyslipidemia, syndrome X, heart failure, hypercholesteremia, cardiovascular disease, type II diabetes mellitus, type I diabetes, insulin resistance hyperlipidemia, obesity, anorexia bulimia, inflammation and anorexia nervosa. Other indications include reduction of scarring and wound healing.

The compounds of the invention may also be used (a) for raising HDL in a subject; (b) for treating Type 2 diabetes, decreasing insulin resistance or lowering blood pressure in a subject; (c) for decreasing LDLc in a subject; (d) for shifting LDL particle size from small dense to normal dense LDL in a subject; (e) for reducing cholesterol absorption or increasing cholesterol excretion in a subject; (f) for reducing the expression of NPC1L1 in a subject; (g) for treating atherosclerotic diseases including vascular disease, coronary heart disease, cerebrovascular disease and peripheral vessel disease in a subject; and (h) for treating inflammatory diseases, including rheumatoid arthritis, asthma, osteoarthritis and autoimmune disease in a subject.

The compounds of the invention may also be used for treating, ameliorating, or preventing a disease or condition selected from the group consisting of obesity, diabetes, hyperinsulinemia, metabolic syndrome X, polycystic ovary syndrome, climacteric, disorders associated with oxidative stress, inflammatory response to tissue injury, pathogenesis of emphysema, ischemia-associated organ injury, doxorubicin-induced cardiac injury, drug-induced hepatotoxicity, atherosclerosis, and hypertoxic lung injury.

The compositions containing the compound(s) described herein can be administered for prophylactic and/or therapeutic treatments. In therapeutic applications, the compositions are administered to a patient already suffering from a disease, condition or disorder mediated, modulated or involving the PPARs, including but not limited to metabolic diseases, conditions, or disorders, as described above, in on amount sufficient to cure or at least partially arrest the symptoms of the disease, disorder or condition. Amounts effective for this use will depend on the severity and course of the disease, disorder or condition, previous therapy, the patient's health status and response to the drugs, and the judgment of the treating physician. It is considered well within the skill of the art for one to determine such therapeutically effective amounts by routine experimentation (e.g., a dose escalation clinical trial).

In prophylactic applications, compositions containing the compounds described herein are administered to a patient susceptible to or otherwise at risk of a particular disease, disorder or condition mediated, modulated or involving the PPARs, including but not limited to metabolic diseases, conditions, or disorders, as described above. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial).

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. When the symptoms have been alleviated to the desired level, treatment can cease. Patients can, however, require intermittent treatment on a long-term basis upon any recurrence of symptoms.

The amount of a given agent that will correspond to such an amount will vary depending upon factors such as the particular compound, disease condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. In general, however, doses employed for adult human treatment will typically be in the range of 0.02-5000 mg per day, preferably 1-1500 mg per day. The desired dose may conveniently be presented in a single dose or as divided doses administered at appropriate intervals, for example as two, three, four or more sub-doses per day.

In certain instances, it may be appropriate to administer at least one of the compounds described herein (or a pharmaceutically acceptable salt, ester, amide, prodrug, or solvate) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of the compound of formula (I) with: (a) statin and/or other lipid lowering drugs for example MTP inhibitors and LDLR upregulators; (b) antidiabetic agents, e.g. metformin, sulfonylureas, or PPAR-gamma, PPAR-alpha and PPAR-alpha/gamma modulators (for example thiazolidinediones such as e.g. Pioglitazone and Rosiglitazone); and (c) antihypertensive agents such as angiotensin antagonists, e.g., telmisartan, calcium channel antagonists, e.g. lacidipine and ACE inhibitors, e.g., enalapril.

In any case, the multiple therapeutic agents (one of which is one of the compounds described herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the liming between the multiple doses may vary from more than zero weeks to less than four weeks.

Suitable routes of administration may, for example, include oral, rectal, transmucosal, pulmonary, ophthalmic or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly into an organ, often in a depot or sustained release formulation. Furthermore, one may administer the drug in a targeted drug delivery system, for example, in a liposome coated with organ-specific antibody. The liposomes will be targeted to and taken up selectively by the organ.

The pharmaceutical compositions of the present invention may be manufactured in a manner that is itself known, e.g., by means of conventional mixing, dissolving, granulating, dragee-making, levigating, emulsifying, encapsulating, entrapping or compression processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For intravenous injections, the agents of the Invention may be formulated in aqueous solutions, preferably in physiologically compatible buffers such as Hanks's solution. Ringer's solution, or physiological saline buffer. For transmucosal administration, penetrants appropriate to the barrier to be permeated are used in the formulation. Such penetrants are generally known in the art. For other parenteral injections, the agents of the invention may be formulated in aqueous or nonaqueous solutions, preferably with physiologically compatible buffers or excipients. Such excipients are generally known in the art.

For oral administration, the compounds can be formulated readily by combining the active compounds with pharmaceutically acceptable carriers or excipients well known in the art. Such carriers enable the compounds of the invention to be formulated as tablets, powders, pills, dragees, capsules, liquids, gels, syrups, elixirs, slurries, suspensions and the like, for oral ingestion by a patient to be treated. Pharmaceutical preparations for oral use can be obtained by mixing one or more solid excipient with one or more compound of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethyl cellulose; or others such as: polyvinylpyrrolidone (PVP or povidone) or calcium phosphate. If desired, disintegrating agents may be added, such as the cross-linked croscarmellose sodium, polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which con be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal or sublingual administration, the compositions may take the form of tablets, lozenges, or gels formulated in conventional manner.

For administration by inhalation, the compounds for use according to the present invention are conveniently delivered in the form of an aerosol spray presentation from pressurized packs or a nebuliser, with the use of a suitable propellant. e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxy methyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. The cosolvent system may be a 10% ethanol, 10% polyethylene glycol 300, 10% polyethylene glycol 40 castor oil (PEG-40 castor oil) with 70% aqueous solution. This cosolvent system dissolves hydrophobic compounds well, and itself produces low toxicity upon systemic administration. Naturally, the proportions of a cosolvent system may be varied considerably without destroying its solubility and toxicity characteristics. Furthermore, the identity of the cosolvent components may be varied: for example, other low-toxicity nonpolar surfactants may be used instead of PEG-40 castor oil, the fraction size of polyethylene glycol 300 may be varied; other biocompatible polymers may replace polyethylene glycol. e.g. polyvinyl pyrrolidone; and other sugars or polysaccharides maybe included in the aqueous solution.

Alternatively, other delivery systems for hydrophobic pharmaceutical compounds may be employed. Liposomes and emulsions are well known examples of delivery vehicles or carriers for hydrophobic drugs. Certain organic solvents such as N-methylpyrrolidone also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms. Salts useful with the compounds of the present invention include, without limitation, calcium acetate, hydrochloric acid, phosphoric acid, sulfuric acid, sodium hydroxide, potassium hydroxide, magnesium acetate, and p-toluenesulfonic acid salts. The salts can be prepared by contacting the compounds of the invention with an appropriate acid, either neat or in a suitable inert solvent, to yield the salt forms of the invention.

All references, patents or applications, U.S. or foreign, cited in the application are hereby incorporated by reference as if written herein.

### General Synthetic Methods for Preparing Compounds

The following schemes can be used to practice the present invention.

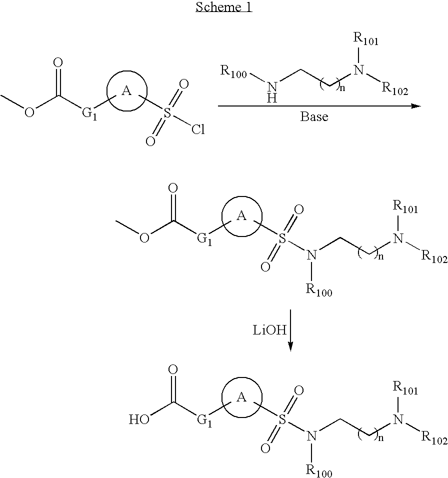

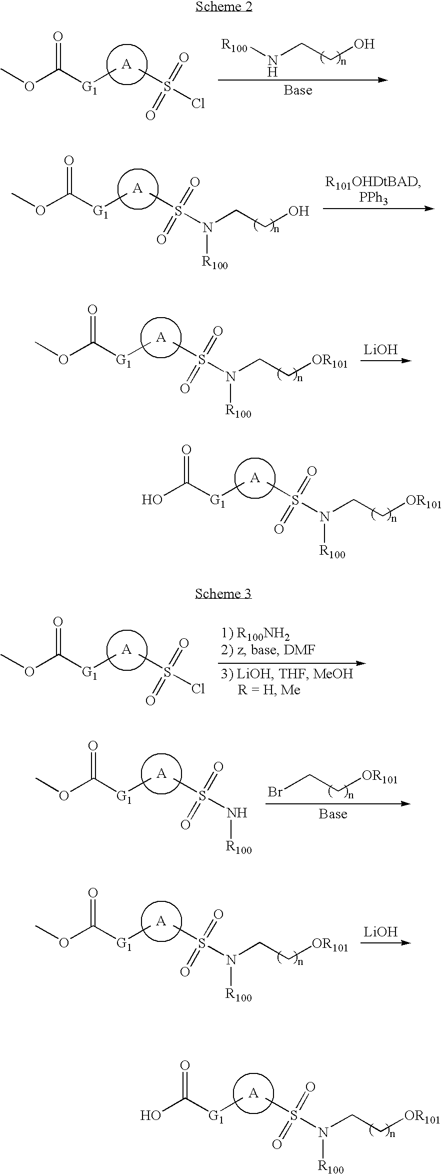

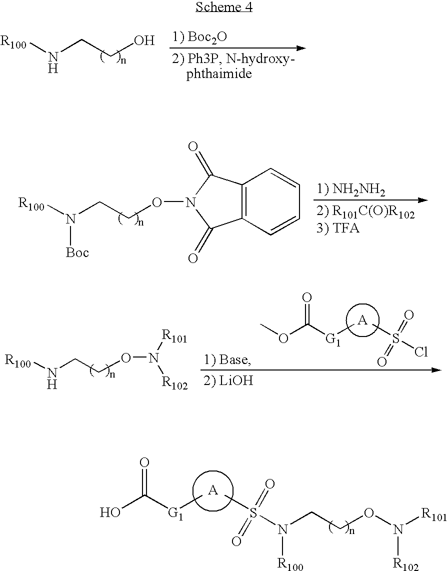

The invention is further illustrated by the following examples.

### EXAMPLE 1

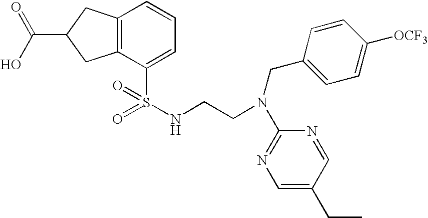

### 4-{2-[(5-Ethyl-pyrimidin-2-yl)-(4-trifluoromethoxy-benzyl}-amino]-ethylsulfamoyl)-indan-2-carboxylic acid

### Step 1

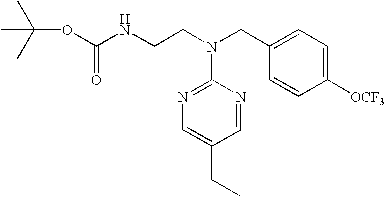

{2-[(5-Ethyl-pyrimidin-2-yl)-(4-trifluoromethoxy-benzyl-amino]-ethyl}-carbamic acid tert-butyl ester: To a solution of 2-chloro-5-ethyl-pyrimidine (77.6 μL, 0.54 mmol) and triethylamine (333 μL, 2.39 mmol) in DMSO (10 mL) was added [2-(4-trifluoromethoxy-benzylamino)-ethyl]-carbamic acid tert-butyl ester (200 mg, 0.60 mmol). The reaction solution was stirred at 150° C. for 24 h. The solution was cooled to room temperature, diluted with water (20 mL) and extracted from ethyl acetate (1×10 mL). The organic solution was dried (Na₂SO₄) and concentrated in vacuo. The residue was purified by silica gel chromatography (0-50% ethyl acetate in hexanes) to afford (2-[(5-ethyl-pyrimidin-2-yl)-(4-trifluoromethoxy-benzyl-amino]-ethyl)-carbamic acid tert-butyl ester (105 mg, 40%) as a yellow oil. ¹H NMR (400 MHz, CDCl₃) δ 8.77 (s, 2H), 7.25 (d, 2H), 7.12 (d, 2H), 5.20-5.10 (m, 1H), 4.58 (s, 2H), 3.72-3.60 (m, 2H), 3.40-3.30 (m, 2H), 2.48 (q, 2H), 1.38 (s, 9H), 1.20 (t, 3H): LCMS 441.5 (M+1)⁺.

### Step 2

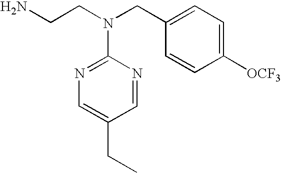

N¹-(5-Ethyl-pyrimidin-2-yl)-N¹-(4-trifluoromethoxy-benzyl)-ethane-1,2-diamine: To a solution of 20% trifluoroacetic acid (2 mL) in methylene chloride (10 mL) was added (2-[(5-ethyl-pyrimidin-2-yl)-{4-trifluoromethoxy-benzyl-amino]-ethyl}-carbamic acid tert-butyl ester (100 mg, 0.28 mmol). The reaction solution was stirred at room temperature for 3 h. The solution was concentrated in vacuo, diluted with ethyl acetate and washed with 1N NaOH. The organic solution was dried (Na₂SO₄) and concentrated to provide N¹-(5-ethyl-pyrimidin-2-yl)-N¹-(4-trifluoromethoxy-benzyl)-ethane-1,2-diamine (73 mg, 95%) as a yellow oil. ¹H NMR (400 MHz, CD₃OD) δ 8.30 (s, 2H), 7.36 (d, 2H), 7.23 (d, 2H), 4.96 (s, 2H), 3.86 (t, 2H), 3.18 (t, 2H), 2.55 (q, 2H), 1.23 (t, 3H); LCMS 341.5 (M+1)⁺.

### Step 3

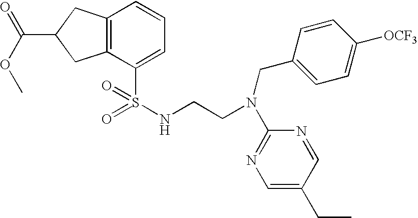

4-{2-[(5-Ethyl-pyrimidin-2-yl)-(4-trifluoromethoxy-benzyl)-amino]-ethylsulfamoyl)-indan-2-carboxylic acid methyl ester: To a solution of 4-chlorosulfonyl-indan-2-carboxylic acid methyl ester (72 mg, 0.26 mmol) and potassium carbonate (121 mg, 0.88 mmol) in acetonitrile (5 mL) was added N¹-(5-ethyl-pyrimidin-2-yl)-N¹-(4-trifluoromethoxy-benzyl)-ethane-1,2-diamine (100 mg, 0.29 mmol). The solution was stirred at 50° C. for 4 h. The reaction mixture was concentrated in vacuo, diluted with ethyl acetate, washed with 1N NaOH and concentrated in vacuo. The residue was purified by silica gel chromatography (0-50% ethyl acetate in hexanes) to afford 4-{2-[(5-ethyl-pyrimidin-2-yl)-(4-trifluoromethoxy-benzyl)-amino]-ethylsulfamoyl)-indan-2-carboxylic acid methyl ester (101 mg, 60%) as a tan oil. ¹H NMR (400 MHz, CD₃OD) δ 8.50 (s, 2H), 7.60 (d, 1H), 7.46 (d, 1H), 7.44-7.36 (m, 2H), 7.35-7.30 (m, 1H), 7.28-7.24 (m, 2H), 5.01 (s, 2H), 3.80 (t, 2H), 3.66 (s, 3H), 3.58-3.34 (m, 2H), 3.28-3.18 (m, 3H), 2.67 (q, 2H), 1.34-1.20 (m, 5H); LCMS 579.5 (M+1)⁺.

### Step 4

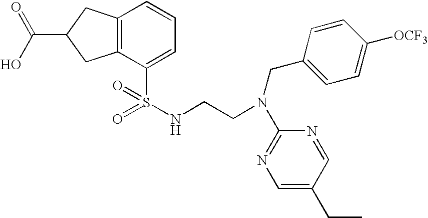

4-{2-[5-Ethyl-pyrimidin-2-yl)-(4-trifluoromethoxy-benzyl}-amino]-ethylsulfamoyl)-indan-2-carboxylic acid: To a solution of 1N LiOH (200 mg, 0.53 mmol) in THF (4 mL) and methanol (1 mL) was added 4-{2-[(5-ethyl-pyrimidin-2-yl)-(4-trifluoromethoxy-benzyl)-amino]-ethylsulfamoyl)-indan-2-carboxylic acid methyl ester (100 mg, 0.18 mmol). The solution was stirred at room temperature for 3 h. The reaction mixture was concentrated in vacuo, diluted with ethyl acetate, washed with 1N HCl, and concentrated in vacuo. The residue was purified by silica gel chromatography (0-20% MeOH in methylene chloride) to afford 4-{2-[(5-ethyl-pyrimidin-2-yl)-(4-trifluoromethoxy-benzyl)-amino]-ethylsulfamoyl)-indan-2-carboxylic acid (85 mg, 88%) as a tan solid. ¹H NMR (400 MHz, CD₃OD) δ 8.50 (s, 2H), 7.60 (d, 1H), 7.46 (d, 1H), 7.44-7.36 (m, 2H), 7.35-7.30 (m, 1H), 7.28-7.24 m, 2H), 5.01 (s, 2H), 3.80 (t, 2H), 3.58-3.34 (m, 2H), 3.28-3.18 (m, 3H), 2.67 (q, 2H), 134-1.20 (m, 5H): LCMS 565.5 (M+1)⁺.

### EXAMPLE 2

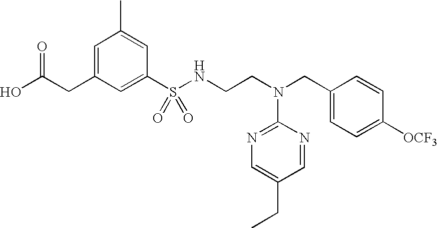

(3-{2-[(Ethyl-pyrimidin-2-yl)-(4-trifluoromethoxy-benzyl)-amino]-ethylsulfamoyl)-5-methyl-phenyl)-acetic acid: The compound (3-{2-[(5-ethyl-pyrimidin-2-yl)-(4-trifluoromethoxy-benzyl)-amino]-ethylsulfamoyl)-5-methyl-phenyl)-acetic acid was prepared according to the procedure outlined in Example 1 using (3-chlorosulfonyl-5-methyl-phenyl)-acetic acid methyl ester. ¹H NMR (400 MHz, CD₃OD) δ 8.55 (s, 2H), 7.54 (d, 2H), 7.42 (d, 2H), 7.37 (s, 1H), 7.28 (d, 2H), 5.05 (s, 2H), 3.84 (t, 2H), 3.72-3.66 (m, 2H), 3.30-3.20 (m, 2H), 2.69 (q, 2H), 2.40 (s, 3H), 1.38-1.20 (m, 3H); LCMS 553.5 (M+1)⁺.

### EXAMPLE 3

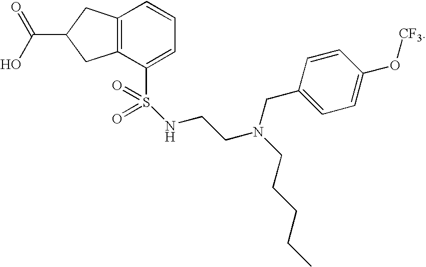

### 4-{2-[Pentyl-(4-trifluoromethoxy-benzyl)-amino]-ethylsulfamoyl}-indan-2-carboxylic acid

### Step 1

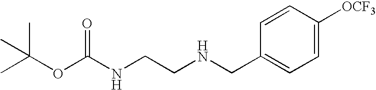

[2-(4-Trifluoromethoxy-benzylamino)-ethyl]-carbamic acid tert-butyl ester: To a solution of 4-(trifluoromethoxy)-benzaldehyde (237 mg, 1.25 mmol) in methylene chloride (30 mL) was added N-(2-aminoethyl)carbamic acid tert-butyl ester (200 mg, 1.25 mmol). After 1 h sodium triacetoxy borohydride (527 mg, 2.50 mmol) was added and the reaction mixture was stirred at room temperature for 4 h. The reaction mixture was concentrated, diluted with ethyl acetate, washed with 1N NaOH and concentrated in vacuo. The residue was purified by silica gel chromatography (0-50% ethyl acetate in hexanes) to afford [2-(4-trifluoromethoxy-benzylamino)-ethyl]-carbamic acid tert-butyl ester (213 mg, 88%) as a clear oil. ¹H NMR (400 MHz, CDCl₃) δ 7.34 (d, 2H), 7.16 (d, 2H), 4.90 (m, 2H), 3.80 (s, 2H), 3.28-3.10 (m, 2H), 2.86-2.70 (m, 2H), 1.45 (s, 9H); LCMS 335.5 (M+1)⁺.

### Step 2

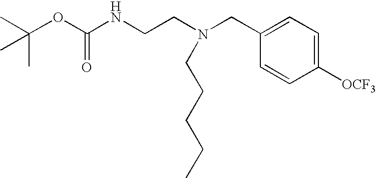

{2-[Pentyl-(4-trifluoromethoxy-benzyl)-amino]-ethyl}-carbamic acid tert-butyl ester: To a solution of valeraldehyde (63 mg, 0.60 mmol) in methylene chloride (30 mL) was added [2-(4-trifluoromethoxy-benzylamino)-ethyl]-carbamic acid tert-butyl ester (200 mg, 0.60 mmol). After 1 h, sodium triacetoxy borohydride (252 mg, 1.19 mmol) was added and the mixture was stirred at room temperature for an additional 4 h. The reaction mixture was concentrated in vacuo, diluted with ethyl acetate, washed with 1N NaOH and concentrated in vacuo. The residue was purified by silica gel chromatography (0-50% ethyl acetate in hexanes) to afford (2-[pentyl-(4-trifluoromethoxy-benzyl)-amino]-ethyl}-carbamic acid tert-butyl ester (217 mg, 95%) as a clear oil. ¹H NMR (400 MHz. CDCl₃) δ 7.31-7.28 (m, 2H), 7.18-7.10 (m, 2H), 4.88-4.70 (m, 1H), 3.54 (s, 2H), 3.20-3.00 (m, 2H), 2.60-2.52 (m, 2H), 2.50-2.45 (m, 2H), 1.50-1.35 (m, 11H), 1.30-1.20 (m, 4H), 0.82 (t, 3H); LCMS 405.5 (M+1)⁺.

### Step 3

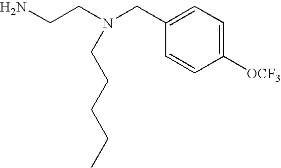

N¹-Pentyl-N¹-(4-trifluoromethoxy-benzyl)-ethane-1,2-diamine: A solution of {2-[pentyl-(4-trifluoromethoxy-benzyl)-amino]-ethyl}-carbamic acid tert-butyl ester (130 mg, 0.32 mmol) and 20% trifluoroacetic acid (2 mL) in dichloromethane (10 mL) was stirred at room temperature for 3 h. The reaction mixture was concentrated/n vacuo, diluted with ethyl acetate and extracted with 1N NaOH. The organic solution was dried (Na₂SO₄) and concentrated in vacuo to provide N¹-pentyl-N¹-(4-trifluoromethoxy-benzyl)-ethane-1,2-diamine (88 mg, 95%) as a clear oil. ¹H NMR (400 MHz, CD₃OD) δ 7.31 (d, 2H), 7.19 (d, 2H), 3.54 (s, 2H), 2.60-2.52 (m, 4H), 2.50-2.45 (m, 2H), 1.60-1.55 (m, 2H), 1.30-1.20 (m, 4H), 0.82 (t, 3H); LCMS 305.5 (M+1)⁺.

### Step 4

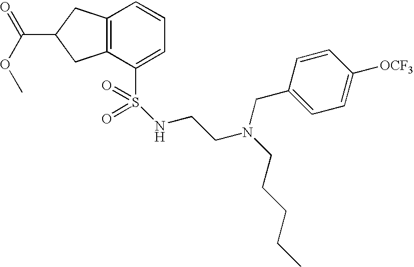

4-{2-[Pentyl-(4-trifluoromethoxy-benzyl)-amino]-ethylsulfamoyl}-indan-2-carboxylic acid methyl ester: A solution of 4-chlorosulfonyl-indan-2-carboxylic acid methyl ester (24 mg, 0.088 mmol), pentyl-N¹-(4-trifluoromethoxy-benzyl)-ethane-1,2-diamine (30 mg, 0.098 mmol), and potassium carbonate (41 mg, 0.30 mmol) in acetonitrile (30 mL) was heated at 50° C. for 4 h. The reaction mixture was concentrated in vacuo, diluted with ethyl acetate, washed with 1N NaOH and concentrated in vacuo. The residue was purified by silica gel chromatography (0-50% ethyl acetate in hexanes) to afford 4-{2-[pentyl-(4-trifluoromethoxy-benzyl)-amino]-ethylsulfamoyl-indan-2-carboxylic acid methyl ester (32 mg, 60%) as a clear oil. ¹H NMR (400 MHz, CDCl₃) δ 7.68 (d, 1H), 7.42 (d, 1H), 7.32-7.24 (m, 3H), 7.20-7.16 (m, 2H), 5.20-5.00 (m, 1H), 3.74 (s, 3H), 3.50-3.46 (m, 2H), 3.40-3.24 (m, 4H), 2.98 (t, 2H), 2.60-2.50 (m, 2H), 2.34 (t, 2H), 1.46-1.38 (m, 2H), 1.34-1.14 (m, 5H), 0.88 (t, 3H); LCMS 543.5 (M+1)⁺.

### Step 5

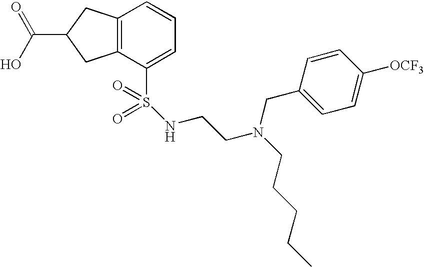

4-{2-[Pentyl-(4-trifluoromethoxy-benzyl)-amino]-ethylsulfamoyl}-indan-2-carboxylic acid: To a solution of 4-{2-[pentyl-(4-trifluoromethoxy-benzyl)-amino]-ethylsulfamoyl}-indan-2-carboxylic acid methyl ester (30 mg, 0.056 mmol) in THF (4 mL), MeOH (1 mL) was added 1N LiOH (200 μL, 0.16 mmol). The reaction solution was stirred at room temperature for 3 h. The reaction mixture was concentrated in vacuo, diluted with ethyl acetate, washed with 1N HCl and concentrated in vacuo. The residue was purified by silica gel chromatography (0-20% MeOH in methylene chloride) to afford 4-{2-[pentyl-(4-trifluoromethoxy-benzyl)-amino]-ethylsulfamoyl}-indan-2-carboxylic acid (27 mg, 88%) as a clear oil. ¹H NMR (400 MHz, CD₃OD) δ 7.72-7.64 (m, 3H), 7.51 (d, 1H), 7.44-7.323 (m, 3H), 4.52-4.40 (m, 2H), 3.72 (s, 2H), 3.58-3.50 (m, 2H), 3.48-3.38 (m, 1H), 3.34-3.14 (m, 4H), 1.86-1.70 (m, 2H), 1.44-1.26 (m, 6H), 0.92 (t, 3H); LCMS 529.5 (M+1)⁺.

### EXAMPLE 4

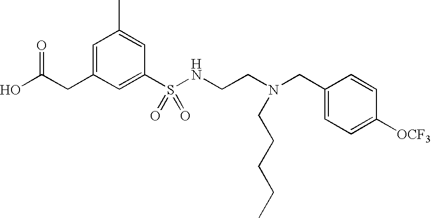

(3-Methyl-5-{2-[pentyl-(4-trifluoromethoxy-benzyl)-amino]-ethylsulfamoyl}-acetic acid: The compound (3-methyl-5-(2[pentyl-(4-trifluoromethoxy-benzyl)amino]-ethylsulfamoyl-acetic acid was prepared according 10 the procedure outlined in Example 3 (3-chlorosulfonyl-5-methyl-phenyl)-acetic acid methyl ester. ¹H NMR (400 MHz, CD₃OD) δ 7.72-7.66 (m, 2H), 7.63-7.58 (m, 2H), 7.45-7.38 (m, 3H), 4.48 (s, 2H), 3.71 (s, 2H), 3.32-3.14 (m, 4H), 2.46 (s, 3H), 1.84-1.72 (m, 2H), 1.44-1.26 (m, 6H), 0.95 (t, 3H); LCMS 517.5 (M+1)⁺.

### EXAMPLE 5

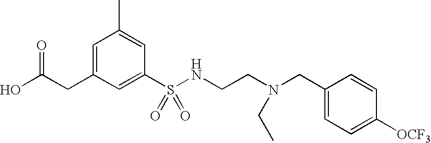

(3-{2-[Ethyl-(4-trifluoromethoxy-benzyl}-amino]-ethylsulfamoyl)-5-methyl-phenyl)-acetic acid: The compound (3-(2-[ethyl-(4-trifluoromethoxy-benzyl)-amino]-ethylsulfamoyl-5-methyl-phenyl)-acetic acid was prepared according to the procedure outlined in Example 3 using acetaldehyde and (3-chlorosulfonyl-5-methyl-phenyl)-acetic acid methyl ester. ¹H NMR (400 MHz, CD₃OD) δ 7.74-7.66 (m, 2H), 7.63 (d, 2H), 7.45-7.38 (m, 3H), 4.48 (s, 2H), 3.71 (s, 2H), 3.34-3.20 (m, 6H), 2.42 (s, 3H), 1.38 (1.3H); LCMS 475.5 (M+1)⁺.

### EXAMPLE 6

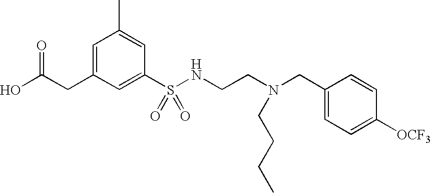

(3-{2-[Butyl-(4-trifluoromethoxy-benzyl)-amino]-ethylsulfamoyl}-5-methyl-phenyl)-acetic acid: The compound (3-{2-(butyl-(4-trifluoromethoxy-benzyl)-amino)-ethylsulfamoyl}-5-methyl-phenyl)-acetic acid was prepared according to the procedure outlined in Example 3 using butyraldehyde and (3-chlorosulfonyl-5-methyl-phenyl)-acetic acid methyl ester. ¹HNMR (400 MHz, CD₃OD) δ 7.72-7.66 (m, 2H), 7.63-7.58 (m, 2H), 7.45-7.38 (m, 3H), 4.48 (s, 2H), 3.71 (s, 2H), 3.32-3.14 (m, 4H), 3.10-3.02 (m, 2H), 2.46 (s, 3H), 1.84-1.72 (m, 2H), 1.44-1.26 (m, 2H), 0.95 (t, 3H); LCMS 503.5 (M+1)⁺.

### EXAMPLE 7

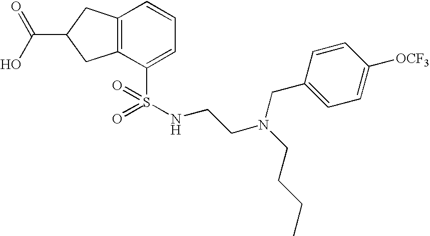

4-{2-[Butyl-(4-trifluoromethoxy-benzyl)-amino]-ethylsulfamoyl}-indan-2-carboxylic acid: The compound 4-{2-[butyl-(4-trifluoromethoxy-benzyl)-amino]-ethylsulfamoyl}-indan-2-carboxylic acid was prepared according to the procedure outlined in Example 3 using butyraldehyde. ¹H NMR (400 MHz, CD₃OD) δ 7.72-7.64 (m, 3H), 7.51 (d, 1H), 7.44-7.323 (m, 3H), 4.48 (s, 2H), 3.58-3.50 (m, 2H), 3.48-3.38 (m, 1H), 334-3.24 (m, 6H), 3.18-3.10 (m, 3H), 1.86-1.70 (m, 2H), 1.44-1.26 (m, 2H), 0.98 (t, 3H); LCMS 515.5 (M+1)⁺.

### EXAMPLE 8

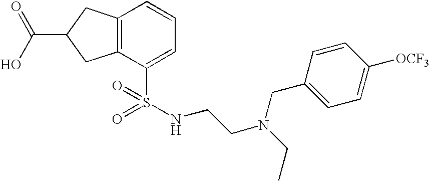

4-{2-[Ethyl-(4-(trifluoromethoxy-benzyl)-amino]-ethylsulfamoyl}-indan-2-carboxylic acid: The compound 4-{2-[ethyl-(4-trifluoromethoxy-benzyl)-amino]-ethylsulfamoyl}-indan-2-carboxylic acid was prepared according to the procedure outlined in Example 3 using acetaldehyde. ¹H NMR (400 MHz, CD₃OD) δ7.72-7.64 (m, 3H), 7.51 (d, 1H), 7.44-7.323 (m, 3H), 4.46 (s, 2H), 3.58-3.50 (m, 2H), 3.48-3.38 (m, 2H), 3.34-3.20 (m, 7H), 1.37 (t, 3H); LCMS 487.5 (M+1)⁺.

### EXAMPLE 9

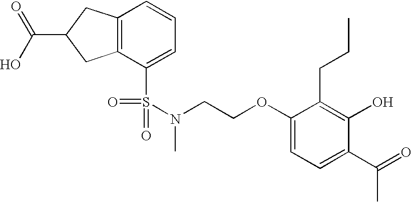

### 4-(N-(2-(4-Acetyl-3-hydroxy-2-propylphenoxy)ethyl)-N-methylsulfamoyl)-2,3-dihydro-1H-indene-2-carboxylic acid

### Step 1

Methyl 4-(N-(2-hydroxyethyl)-N-methylsulfamoyl-2,3-dihydro-1H-indene-2-carboxylate: 2-(Methylamino)ethanol (0.35 mL, 4.33 mmol) and DMAP (20 mg, 0.16 mmol) were sequentially added to a solution of methyl 4-(chlorosulfonyl)-2,3-dihydro-1H-indene-2-carboxylate (900 mg, 3.28 mmol), triethylamine (1.5 mL, 11 mmol), and THF (30 mL) at room temperature under N₂. After 1 h, the reaction was poured into 1N HCl (100 mL) and extracted with dichloromethane (100 mL×2). The combined organic extracts were combined, dried, filtered, concentrated in vacuo, and purified by silica gel chromatography (3:2→1:4:hexanes:ethyl acetate) to give methyl 4-(N-(2-hydroxyethyl)-N-methylsulfamoyl)-2,3-dihydro-1H-indene-2-carboxylate.

### Step 2

Methyl 4-(N-(2-(4-acetyl-3-hydroxy-2-propylphenoxy)ethyl)-N-methylsulfamoyl)-2,3-dihydro-1H-indene-2-carboxylate: Triphenylphosphine (265 mg, 1 mmol) and di-tert-butylazodicarboxylate (230 mg, 1 mmol) were sequentially added to a solution of methyl 4-(N-(2-hydroxyethyl)-N-methylsulfamoyl)-2 ]-dihydro-1H-indene-2-carboxylate (157 mg, 0.5 mmol), 1-(2,4-dihydroxy-3-propylphenyl)ethanone (150 mg, 0.77 mmol) and THF (4 mL). After 22 h, the reaction was concentrated and purified by silica gel chromatography (4:1→3:2; hexanes:ethyl acetate) to give methyl 4-(N-(2-(4-acetyl-3-hydroxy-2-propylphenoxy)ethyl)-N-methylsulfamoyl)-2,3-dihydro-1H-indene-2-carboxylate: MS (ESI): 490.0 (M+H).

### Step 3

4-(N-(2-(4-Acetyl-3-hydroxy-2-propylphenoxy)ethyl)-N-methylsulfamoyl)-2,3-dihydro-1H-indene-2-carboxylic acid: The title compound was prepared from methyl 4-(N-(2-(4-acetyl-3-hydroxy-2-propylphenoxy)ethyl)-N-methylsulfamoyl)-2,3-dihydro-1H-indene-2-carboxylate following the procedure outlined in Example 1, Step 4. ¹HNMR (400 MHz. DMSO-d6): δ 12.81 (s, 1H), 12.39 (brs, 1H), 7.79 (d, 1H), 7.56 (d, 1H), 7.52 (d, 1H), 7.37 (t, 1H), 6.62 (d, 1H), 4.2) (t, 2H), 3.50 (t, 2H), 3.46-3.11 (m, 5H), 2.84 (s, 3H), 2.57 (s, 3H), 2.53 (t, 2H), 1.43 (m, 2H), 0.85 (t, 3H): MS (ESI): 476.0 (M+H).

### EXAMPLE 10

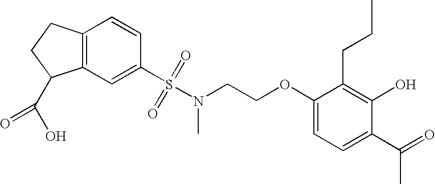

6-(N-(2-(4-Acetyl-3-hydroxy-2-propylphenoxy)ethyl)-N-methylsulfamoyl)-2-dihydro-1H-indene-1-carboxylic acid: The title compound was prepared from methyl 6-(chlorosulfonyl)-2,3-dihydro-1H-indene-1-carboxylate following the procedure outlined in Example 9. ¹H NMR (400 MHz, DMSO-d6): δ 12.81 (s, 1H), 12.61 (brs, 1H), 7.81 (d, 1H), 7.75 (s, 1H), 7.65 (d, 1H), 7.49 (d, 1H), 6.63 (d, 1H), 4.22 (t, 2H), 4.10 (t, 1H), 3.38 (m, 2H), 3.08-2.88 (m, 2H), 2.80 (s, 3H), 2.59 (s, 3H), 2.53 (t, 2H), 239-2.25 (m, 2H), 1.44 (m, 2H), 0.86 (t, 3H); MS (ESI): 476.0 (M+H).

### EXAMPLE 11

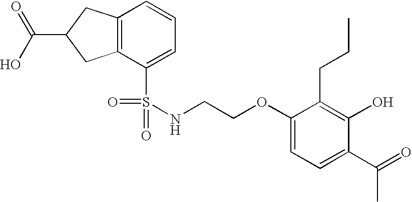

### 4-(N-(2-(4-Acetyl-3-hydroxy-2-propylphenoxy)ethyl)sulfamoyl)-2,3-dihydro-1H-indene-2-carboxylic acid

### Step 1

Methyl 4-sulfamoyl-2,3-dihydro-1H-indene-2-carboxylate: Ammonia (3.5 mL, 2M in methanol, 7 mmol) was added to a solution of methyl 4-(chlorosulfonyl)-2,3-dihydro-1H-indene-2-carboxylate (825 mg, 3 mmol) and dichloromethane (15 mL) at room temperature. After 1.5 h, the reaction was concentrated in vacuo and purified by silica gel chromatography (7:3→2:3; hexanes:ethyl acetate) to give methyl 4-sulfamoyl-2,3-dihydro-1H-indene-2-carboxylate: MS (ESI): 255.9 (M+H).

### Step 2

Methyl 4-(N-(2-(4-acetyl-3-hydroxy-2-propylphenoxy)ethyl)sulfamoyl)-2,3-dihydro-1H-indene-2-carboxylate: A mixture of methyl 4-sulfamoyl-2,3-dihydro-1H-indene-2-carboxylate (270 mg, 1.1 mmol), 1-(4-(2-bromoethoxy)-2-hydroxy-3-propylphenyl)ethanone (320 mg, 1.1 mmol), cesium carbonate (550 mg, 1.7 mmol) and DMF (4 mL) was stirred at room temperature under N₂. After 14 h, the reaction was diluted with 0.1 N HCl (40 mL) and extracted with dichloromethane (40 mL×2). The combined organic extracts were dried, filtered, concentrated, and purified by silica gel chromatography (4:1→1:2; hexanes:ethyl acetate). Further purification by reverse-phase HPLC (1:1→0:1; water:acetonitrile) gave methyl 4-(N-(2-(4-acetyl-3-hydroxy-2-propylphenoxy)ethyl)sulfamoyl)-2,3-dihydro-1H-indene-2-carboxylate: MS (ESI): 476.0 (M+H).

### Step 3

4-(N-(2-(4-Acetyl-3-hydroxy-2-propylphenoxy)ethyl)sulfamoyl)-2,3-dihydro-1H-indene-2-carboxylic acid: The title compound was prepared from methyl 4-(N-(2-(4-acetyl-3-hydroxy-2-propylphenoxy)ethyl)sulfamoyl)-2,3-dihydro-1H-indene-2-carboxylate following the procedure outlined in Example 1, Step 4. ¹H NMR (400 MHz, DMSO-d6): δ 12.81 (s, 1H), 7.96 (t, 1H), 7.76 (d, 1H), 7.60 (d, 1H), 7.47 (d, 1H), 7.33 (t, 1H), 6.52 (d, 1H), 4.03 (m, 2H), 3.56-3.26 (m, 3H), 3.16 (m, 4H), 2.57 (s, 3H), 2.50 (m, 2H), 1.42 (m, 2H), 0.83 (t, 3H); MS (ESI): 461.9 (M+H).

### EXAMPLE 12

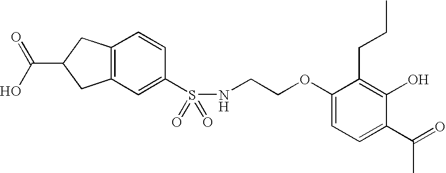

5-(N-(2-(4-Acetyl-3-hydroxy-2-propylphenoxy)ethyl)sulfamoyl)-2,3-dihydro-1H-indene-2-carboxylic acid: The title compound was prepared from methyl 5-(chlorosulfonyl)-2,3-dihydro-1H-indene-2-carboxylate following the procedure outlined in Example 11. ¹H NMR (400 MHz. DMSO-d6): δ 12.81 (s, 1H), 7.87 (t, 1H), 7.77 (d, 1H), 7.64 (s, 1H), 7.60 (d, 1H), 7.39 (d, 1H), 6.53 (d, 1H), 4.04 (t, 2H), 3.31 (m, 1H), 3.15 (m, 6H), 2.57 (s, 3H), 2.53 (t, 2H), 1.43 (m, 2H), 0.84 (t, 3H); MS (ESI): 462.0 (M+H).

### EXAMPLE 13

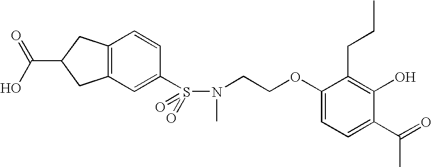

### 5-N-(2-(4-Acetyl-3-hydroxy-2-propylphenoxy)ethyl)-N-methylsulfamoyl)-2,3-dihydro-1H-indene-2-carboxylic acid

### Step 1

Methyl 5-(N-methylsulfamoyl)-2,3-dihydro-1H-indene-2-carboxylate: Methylamine (9 mL, 2M in THF, 18 mmol) was added to a solution of methyl 5-(chlorosulfonyl)-2,3-dihydro-1H-indene-2-carboxylate (1.6 g, 5.8 mmol) and dichloromethane (20 mL) at room temperature. After 15 min, The reaction was concentrated and purified by silica gel chromatography (7:3→2:3; hexanes:ethyl acetate) to give methyl 5-(N-methylsulfamoyl)-2,3-dihydro-1H-indene-2-carboxylate.

### Step 2

5-(N-(2-(4-Acetyl-3-hydroxy-2-propylphenoxy)ethyl)-N-methylsulfamoyl)-2,3-dihydro-1H-indene-2-carboxylic acid: The title compound was prepared from methyl 5-(N-methylsulfamoyl)-2,3-dihydro-1H-indene-2-carboxylate following the procedure outlined in Example 11. ¹H NMR (400 MHz, DMS-d6): δ 12.81 (s, 1H), 7.81 (d, 1H), 7.65 (s, 1H), 7.59 (d, 1H), 7.45 (d, 1H), 6.63 (d, 1H), 4.21 (t, 2H), 3.41 (t, 2H), 3.36-3.12 (m, 5H), 2.81 (s, 3H), 2.59 (s, 3H), 2.53 (t, 2H), 1.44 (m, 2H), 0.86 (t, 3H); MS (ESI): 476.0 (M+H).

### EXAMPLE 14

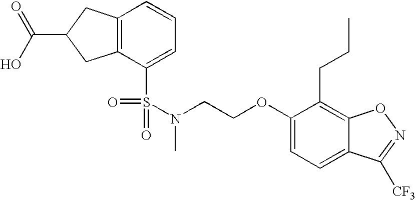

4-(N-methyl-N-(2-(7-propyl-3-(trifluoromethyl)benzo[d]isoxazol-6-yloxy)ethyl)sulfamoyl)-2,3-dihydro-1H-indene-2-carboxylic acid: The compound 4-(N-methyl-N-(2-(7-propyl-3-(trifluoromethyl)benzo[d]isoxazol-6-yloxy)ethyl)sulfamoyl)-2,3-dihydro-1H-indene-2-carboxylic acid was prepared from methyl 4-(chlorosulfonyl)-2,3-dihydro-1H-indene-2-carboxylate and 7-propyl-3-(trifluoromethyl)benzo[d]isoxazol-6-ol following the procedure outlined in Example 9. ¹HNMR (400 MHz, DMSO-d6): δ 7.75 (d, 1H), 7.57 (d, 1H), 7.51 (d, 1H), 736 (t, 1H), 734 (d, 1H), 430 (t, 2H), 3.55 (t, 2H), 3.50-3.10 (m, 5H), 2.85 (m, 5H), 1.62 (m, 2H), 0.88 (t, 3H); MS (ESI): 527.5 (M+H).

### EXAMPLE 15

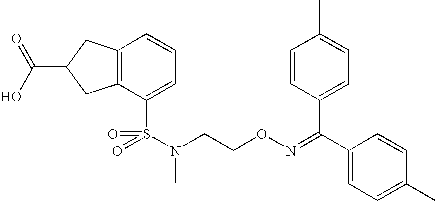

### 4-(N-(2-(di-p-tolylmethyleneaminooxy)ethyl)-N-methylsulfamoyl)-2,3-dihydro-1H-indene-2-carboxylic acid

### Step 1

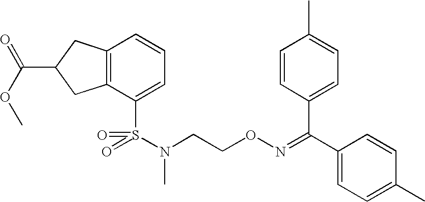

Methyl 4-(N-(di-p-tolylmethyleneaminooxy)ethyl)-N-methylsulfamoyl)-2,3-dihydro-1H-indene-2-carboxylate: 4-[(2-bromo-ethyl)-methyl-sulfamoyl]-indan-2-carboxylic acid methyl ester (101 mg, 0.27 mmol), di-p-tolyl-methanone oxime (90 mg, 0.40 mmol), TBAI (15 mg, 15 mol %) and potassium carbonate (115 mg, 0.83 mmol) were mixed in DMF (3 mL). The reaction was stirred at room temperature for 2 h then heated to 100° C. for 0.5 h. The mixture was poured into water and extracted with ethyl acetate, dried (Na₂SO₄), filtered and concentrated. The product was purified by silica gel chromatography (0-30% EtOAc in Hexanes) to afford methyl 4-(N-(2-(di-p-tolylmethyleneaminooxy)ethyl)-N-methylsulfamoyl)-2,3-dihydro-1H-indene-2-carboxylate (35 mg, 25%) as a clear oil. ¹H NMR (400 MHz, CDCl₃) δ 7.61 (d, 1H), 7.39 (d, 1H), 7.34 (d, 2H), 7.26 (t, 1H), 7.12 (d, 2H), 4.33-4.28 (m, 2H), 3.70 (s, 3H), 3.65-3.61 (m, 1H), 3.53-3.45 (m, 3H), 3.38-3.21 (m, 4H), 2.79 (s, 3H), 2.39 (s, 3H), 2.35 (s, 3H). LCMS: 521.0 (M+1)⁺.

### Step 2

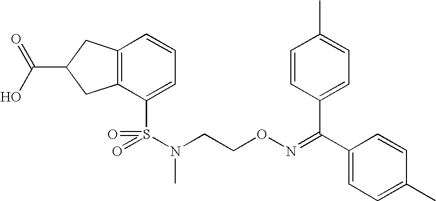

4-(N-(2-(Di-p-tolylmethyleneaminooxy)ethyl)-N-methylsulfamoyl)-2,3-dihydro-1H-indene-2-carboxylic acid: 1 M LiOH (1 mL) was added to methyl 4-(N-(2-(di-p-tolylmethyleneaminooxy)ethyl)-N-methylsulfamoyl)-2,3-dihydro-1H-indene-2-carboxy late (35 mg, 0.067 mmol) in THF (4 mL) and methanol (1 mL). The reaction was stirred for 3 h at room temperature and then quenched with DOWEX 50WX4-50 (H⁺ Form) until the solution is neutral (pH paper). The solution was filtered, concentrated and purified by silica gel chromatography (0-20% MeOH in dichloromethane) to afford 4-(N-(2-(di-p-tolylmethyleneaminooxy)ethyl)-N-methylsulfamoyl)-2,3-dihydro-1H-indene-2-carboxylic acid (19 mg, 56%) as a clear oil. LCMS: 507.0 (M+1)⁺.

### EXAMPLE 16

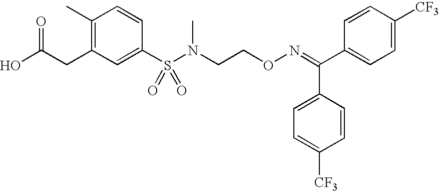

### 2-(2-(N-(2-(bis(4-trifluoromethyl)phenyl)methyleneaminooxy)ethyl)-N-methylsulfamoyl-6-methylphenyl)acetic acid

### Step 1

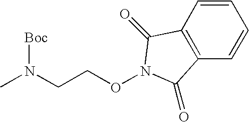

tert-Butyl 2-(1,3-dioxoisoindolin-2-yloxy)ethyl(methyl)carbamate: 2-(Methylamino)ethanol (10.0 g, 133.1 mmol), triethylamine (55 mL, 394.6 mmol), and Boc₂O (18 mL, 78.4 mmol) were mixed in DMF (50 mL). The reaction was stirred at room temperature for 1 h then dry loaded on SiO₂ and purified by flash chromatography to afford tert-butyl 2-hydroxyethyl(methyl)carbamate (10.85 g), tert-Butyl 2-hydroxyethyl(methyl)carbamate (10.85 g, 61.9 mmol) was then dissolved in THF (500 mL). To this solution was then added triphenylphosphine (18.20 g, 69.4 mmol), N-hydroxyphthalimide (12.13 g, 74.4 mmol) and di-tert-butyl azodicarboxylate (19.50 g, 84.7 mmol) at 0° C. The reaction mixture was then stirred for an additional 2.5 h at 0° C., concentrated on SiO₂ and purified by silica gel chromatography (0-50% EtOAc in Hexanes) to afford tert-butyl 2-(1,3-dioxoisoindolin-2-yloxy)ethyl(methyl)carbamate (11.17 g, 56%) as a yellow oil. ¹H NMR (400 MHz, CDCl₃) δ 7.83-7.81 (m, 2H), 7.78-7.73 (m, 2H), 4.40-4.25 (m, 2H), 3.65-3.55 (m, 2H), 3.02 (br s, 3H), 1.44 (br s, 9H).

### Step 2

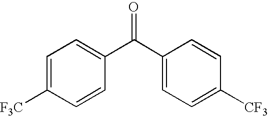

Bis-(4-(trifluromethyl)phenyl)methanone: Co₂(CO)_{g} was added to a solution of 1-iodo-4-(trifluoromethyl)benzene (2.50 g, 9.2 mmol) in CH₃CN (17 mL). Using a microwave reactor (biotage) the reaction was heated to 130° C. for 10 seconds. The reaction was filtered through celite, concentrated, dry loaded on SiO₂ and purified by silica gel chromatography (0-30% EtOAc in Hexanes) to afford bis-(4-(trifluoromethyl)phenyl)methanone (1.13 g, 61%) as an off white solid. ¹H NMR (400 MHz, CDCl₃) δ 7.91 (d, 4H), 7.79 (d, 4H).

### Step 3

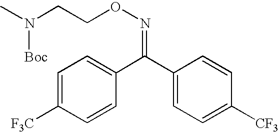

tert-Butyl-2-(bis(4-(trifluoromethyl)phenyl)methyleneaminooxy)ethyl(methyl) carbamate: Hydrazine monohydrate (240 μL, 4.9 mmol) was added to a solution of tert-butyl-2-(1,3-dioxoisoindolin-2-yloxy)ethyl(methyl)carbamate (1.0 g, 3.1 mmol) in EtOH (15 mL) at room temperature. When the deprotection was complete by TLC, the mixture was concentrated to afford an off-white solid which was then extracted with diethyl ether. The ether solution was then filtered and concentrated to provide crude tert-butyl 2-(aminooxy)ethyl(methyl)carbamate (600 mg) as a yellow oil. The crude tert-butyl 2-(aminooxy)ethyl(methyl)carbamate (219 mg, 1.15 mmol) and bis(4-(trifluoromethyl)phenyl)methanone (440 mg, 1.4 mmol) were mixed in MeOH (3 mL). To this mixture was added solid NaOH (6 equiv) which was followed by stirring for 30 min at 70° C. The reaction mixture was diluted with EtOAc and washed with water, brine, dried with sodium sulfate, filtered, concentrated and purified by silica gel chromatography (0-30% EtOAc in Hexanes) to afford tert-butyl-2-(bis(4-(trifluoromethyl)phenyl)methyleneaminooxy)ethyl(methyl) carbamate (270 mg, 48%) as a while solid. ¹H NMR (400 MHz, CDCl₃) δ 7.23-7.71 (m, 2H), 7.62-7.56 (m, 4H), 7.49-7.42 (m, 2H), 4.35-4.25 (m, 2H), 3.55-3.49 (m, 2H), 2.80 (s, 3H), 1.41 (brs, 9H); LCMS: 491.4 (M+1)⁺.

### Step 4

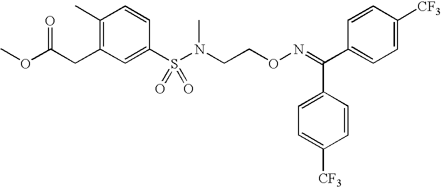

Methyl 2-(5-(N-(2-(bis(4-(trifluoromethyl)phenyl)methyleneaminooxy)ethyl)-N-methylsulfamoyl)-2-(methylphenyl)acetate: A solution of tert-butyl-2-(bis(4(trifluoromethyl)phenyl)methyleneaminooxy)ethyl(methyl)carbamate (127 mg, 0.26 mmol) in 10% TFA/dichloromethane (2.5 mL) was stirred for 1 h. The reaction mixture was concentrated in vacuo and dissolved in THF (2 mL). To this solution was added triethylamine (200 μL, 1.43 mmol), methyl 2-(5-(chlorosulfonyl)-2-methylphenyl)acetate (86 mg, 0.33 mmol) and DMAP (cat). The mixture was stirred at room temperature for 30 min, concentrated in vacuo and purified by silica gel chromatography (0-30% EtOAc in Hexanes) to afford methyl 2-(5-(4-(2-(bis(4-(trifluoromethyl)phenyl)methyleneaminooxy)ethyl)-N-methylsulfamoyl)-2-methylphenyl)acetate (70 mg, 48%) as a clear oil. ¹H NMR (400 MHz, CDCl₃) δ 7.71 (d, 2H), 7.61-7.55 (m, 6H), 7.47 (d, 2H), 7.30 (d, 1H), 4.36 (t, 2H), 3.69 (s, 5H), 3.35 (t, 2H), 2.69 (s, 3H), 2.36 (s, 3H): LCMS: 617.4 (M+1)⁺.

### Step 5

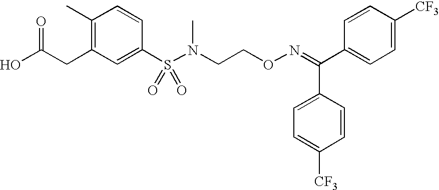

2-(2-(N-(2-(bis(4-Triflouromethyl)phenyl)methyleneaminooxy)ethyl)-n-methylsulfamoyl)-6-methylphenyl)acetic acid: The compound 2-(2-(N-(2-(bis(4-(trifluoromethyl)phenyl)methyleneaminooxy)ethyl)-N-methylsulfamoyl)-6-methylphenyl)acetic acid was prepared from methyl 2-(5-(N-(2-(bis(4-(trifluoromethyl)phenyl)methyleneaminooxy)ethyl)-N-methylsulfamoyl)-2-methylphenyl)acetate according to the procedure outlined in Example 15, Step 2. ¹H NMR (400 MHz, CD₃OD) δ ppm. 7.78 (d, 2H), 7.69-7.63 (m, 5H), 7.58-7.54 (m, 3H), 7.37 (d, 1H), 434 (t, 2H), 3.73 (s, 2H), 3.35 (t, 2H), 2.67 (s, 3H), 2.36 (s, 3H). LCMS: 603.4 (M+1)⁺.

### EXAMPLE 17

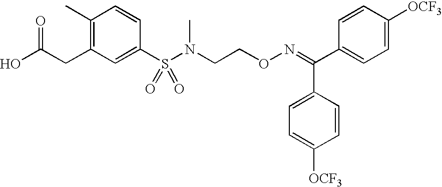

2-{2-(N-(2-(bis(4-(Trifluoromethoxy)phenyl}methyleneaminooxy)ethyl)-N-methylsulfamoyl)-6-methylphenyl)acetic acid: The title compound was prepared following the procedure outlined in Example 16 using bis-(4-(trifluoromethoxy)phenyl)methanone. ¹H NMR (400 MHz, CD₃OD) δ 7.64 (d, 1H), 739-7.54 (m, 3H), 7.47-7.44 (m, 2H), 7.38-7.35 (m, 3H), 7.28 (d, 2H), 430 (t, 2H), 3.73 (s, 2H), 3.35 (t, 2H), 2.67 (s, 3H), 2.37 (s, 3H). LCMS: 635.3 (M+1)⁺.

### EXAMPLE 18

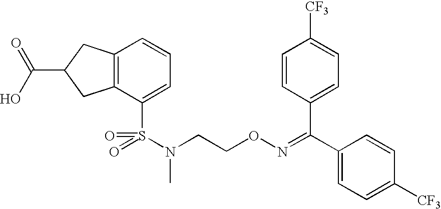

4-(N-(2-(bis(4-(trifluoromethyl)phenyl)methyleneaminooxy)ethyl)-N-methylsulfamoyl)-2,3-dihydro-1H-indene-2-carboxylic acid: The title compound was prepared following the procedure outlined in Example 16 using methyl 4-(chlorosulfonyl)-2,3-dihydro-1H-indene-2-carboxylate. ¹H NMR (400 MHz, CD₃OD) δ 7.78 (d, 2H), 7.69-7.54 (m, 7H), 7.46 (d, 1H), 7.31 (t, 1H), 433 (t, 2H), 3.57-3.44 (m, 4H), 336-3.30 (m, 1H), 3.25-3.23 (m, 2H), 2.74 (s, 3H). LCMS: 615.4 (M+1)⁺.

### EXAMPLE 19

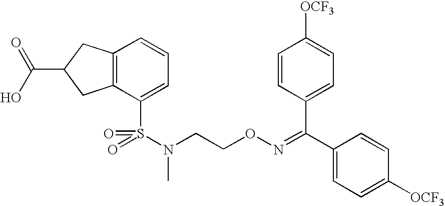

4-(N-(2-(bis(4-(trifluoromethoxy)phenyl)methyleneaminooxy)ethyl)-N-methylsulfamoyl)-2,3-dihydro-1H-indene-2-carboxylic acid: The title compound was prepared following the procedure outlined in Example 16 using bis-(4-(trifluoromethoxy)phenyl)methanone and methyl 4-(chlorosulfonyl)-2,3-dihydro-1H-indene-2-carboxylate. ¹H NMR (400 MHz, CD₃OD) δ 7.59-7.53 (m, 3H), 7.47-7.44 (m, 3H), 7.37-7.26 (m, 5H), 4.30 (t, 2H), 3.58-3.42 (m, 4H), 3.36-3.30 (m, 1H), 3.25-3.23 (m, 2H), 2.74 (s, 3H). LCMS: 647.4 (M+1)⁺.

### EXAMPLE 20

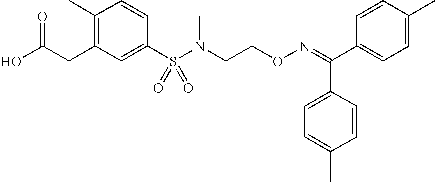

2-(2-(N-(2-(di-p-tolylmethyleneaminooxy)-ethyl-N-methylsulfamoyl)-6-methylphenyl)acetic acid: The title compound was prepared following the procedure outlined in Example 16 using bis-(4-(methyl)phenyl)methanone. ¹H NMR (400 MHz, CD₃OD) δ 7.63 (d, 1H), 7.57 (m, 1H), 7.36 (d, 1H), 7.30 (d, 2H), 7.24-7.13 (m, 6H), 4.24 (t, 2H), 3.72 (s, 2H), 3.32 (t, 2H), 2.66 (s, 3H), 2.38 (s, 3H), 2.36 (s, 3H), 2.34 (s, 3H). LCMS: 495.5 (M+1)⁺.

The compounds in examples 1-20 have been shown to be PPAR modulators by the following assay.

### Biological Activity Assay

Compounds may be screened for functional potency in transient transfection assays in CV-1 cells for their ability to activate the PPAR subtypes (transactivation assay). A previously established chimeric receptor system was utilized to allow comparison of the relative transcriptional activity of the receptor subtypes on the same synthetic response element and to prevent endogenous receptor activation from complicating the interpretation of results. See, for example, Lehmann, J. M.; Moore, L. B.; Smith-Oliver, T. A: Wilkinson, W. O.; Willson, T. M.; Kliewer, S. A., An antidiabetic thiazolidinedione is a high affinity ligand for peroxisome proliferator-activated receptor δ (PPARδ), J. Biol. Chem., 1995, 270, 12953-6. The ligand binding domains for murine and human PPAR-alpha, PPAR-gamma, and PPAR-delta are each fused to the yeast transcription factor GAL4 DNA binding domain. CV-1 cells were transiently transfected with expression vectors for the respective PPAR chimera along with a reporter construct containing four or five copies of the GAL4 DNA binding site driving expression of luciferase. After 8-16 h, the cells are replated into multi-well assay plates and the media is exchanged to phenol-red free DME medium supplemented with 5% delipidated coif serum. 4 hours after replating, cells were treated with either compounds or 1% DMSO for 20-24 hours. Luciferase activity was then assayed with Britelite (Perkin Elmer) following the manufacturer's protocol and measured with either the Perkin Elmer Viewlux or Molecular Devices Acquost (see, for example. Kliewer, S. A., et. al. Cell 1995, 83, 813-819). Rosiglitazone is used as a positive control in the PPARγ assay. Wy-14643 and GW7647 is used as a positive control in the PPARδ assay. GW50156 is used as the positive control in the PPARδ assay.

Examples 1-20 were assayed to measure their biological activity with respect to their efficacy for modulating PPAR-alpha, PPAR-gamma, and PPAR-delta. EC₅₀ values are set forth below in Table 1.

TABLE I
Biological Activity

| | PPAR alpha | PPAR delta | PPAR gamma |
|---|---|---|---|
| | A > 100 μM | A > l00 μM | A > 100 μM |
| | B = 5-100 μM | B = 5-100 μM | B = 5-100 μM |
| Example # | C = <5 μM | C = <5 μM | C = <5 μM |
| 1 | C | C | C |
| 2 | C | C | C |
| 3 | C | C | C |
| 4 | C | C | C |
| 5 | B | C | C |
| 6 | C | C | C |
| 7 | C | C | C |
| 8 | C | C | C |
| 9 | A | C | C |
| 10 | C | C | C |
| 11 | A | C | C |
| 12 | B | B | B |
| 13 | A | A | B |
| 14 | C | C | C |
| 15 | A | C | A |
| 16 | C | C | C |
| 17 | C | C | C |
| 18 | A | C | C |
| 19 | A | C | C |
| 20 | C | C | C |

From the foregoing description, one skilled in the art can easily ascertain the essential characteristics of this invention, and without departing from the spirit and scope thereof, can make various changes and modifications of the invention to adapt it to various usages and conditions.

We claim:

1. A compound having structural Formula (I) 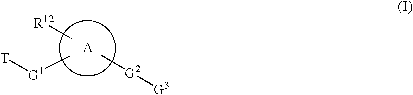
wherein:
A is selected from the group consisting of aryl, heteroaryl, cycloalkyl, and heterocycloalkyl, any of which may be optionally substituted;
R¹² is selected from the group consisting of hydrogen, lower alkyl, lower alkenyl, lower heteroalkyl, and lower alkoxy; R¹² may join together with a carbon atom in G¹ to form a five to eight-membered carbocycle or heterocycle, having structural Formula (II): 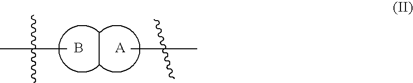
B is a saturated, partially saturated, or unsaturated hydrocarbon chain, optionally containing one or more heteroatoms, to form an optionally substituted five- to eight-membered carbocycle or heterocycle;
T is —C(O)OH;
G¹ is —(CR¹R²)ₙ—;
n is 1 to 4;
R¹ and R² are each independently selected from the group consisting of hydrogen, halogen, lower alkyl, loweralkoxy, and lower perhaloalkyl;
G² is —Y(CR³R⁴)ₚW(CR⁵R⁴)ₘ—,
Y is S, —SO₂N(R⁵)— or NR⁶;
W is O or —NR⁶;
p is 2;
m is 0, 1 or 2;
R³ and R⁴ are each independently selected from the group consisting of hydrogen, halogen, hydroxy, optionally substituted lower alkyl, optionally substituted lower alkoxy, optionally substituted heteroalkyl, optionally substituted cycloalkyl, lower perhaloalkyl, lower perhaloalkoxy, nitro, cyano, NH₂, and —C(O)OR¹¹;
R¹¹ is selected from the group consisting of hydrogen and optionally substituted lower alkyl;
R⁵ and R⁶ are each independently selected from the group consisting of hydrogen, optionally substituted lower alkyl, optionally substituted lower alkenyl, optionally substituted lower alkynyl, optionally substituted heteroalkyl, optionally substituted aryl, and optionally substituted heteroaryl;
G³ is selected from the group consisting of optionally substituted aryl, optionally substituted heteroaryl, optionally substituted cycloalkyl, optionally substituted cycloheteroalkyl, and —N═C(R⁷R⁸); and
R⁷ and R⁸ are each individually selected from the group consisting of hydrogen, optionally substituted aryl, optionally substituted heteroaryl, optionally substituted cycloalkyl, and optionally substituted cycloheteroalkyl.

2. The compound as recited in Claim 1 wherein:
R³ and R⁴ are each hydrogen; and
Y is —SO₂N(R⁵)—.

3. The compound as recited in claim 2 wherein:
A is optionally substituted phenyl;
R¹² is hydrogen; and
R¹ and R² are each independently selected from the group consisting of hydrogen, methyl, ethyl, and propyl.

4. The compound as recited in claim 3 wherein W is —NR⁶.

5. The compound as recited in claim 4 wherein m is 1.

6. The compound as recited in claim 5 wherein G³ is optionally substituted aryl.

7. The compound as recited in claim 6 wherein said aryl is optionally substituted with one or more of the following: halogen, perhaloalkyl, and perhaloalkoxy.

8. The compound as recited in claim 7 wherein said aryl is substituted with perhaloalkoxy.

9. The compound as recited in claim 8 wherein said perhaloalkoxy is trifluoromethoxy.

10. The compound as recited in claim 9 wherein said trifluoromethoxy substitutes said aryl in the para position.

11. The compound as recited in claim 3 wherein:
W is O;
m is 0; and
G₃ is optionally substituted aryl.

12. The compound as recited in claim 3 wherein:
W is O;
m is 0; and
G₃ is optionally substituted heteroaryl.

13. The compound as recited in claim 3 wherein:
W is O;
m is 0; and
G³ is —N═C(R⁷R⁸).

14. The compound as recited in claim 13 wherein at least one of R⁷ and R⁸ is optionally substituted aryl.

15. The compound as recited in claim 14 wherein both R⁷ and R⁸ are optionally substituted aryl.

16. The compound as recited in claim 2 wherein:
A is optionally substituted phenyl;
R¹² joins together with a carbon atom in G¹ to form a five to eight-membered carbocycle or heterocycle;
R¹ and R² are each independently selected from the group consisting of hydrogen, methyl, ethyl, and propyl.

17. The compound as recited in claim 16 having a structural Formula selected from the group consisting of: 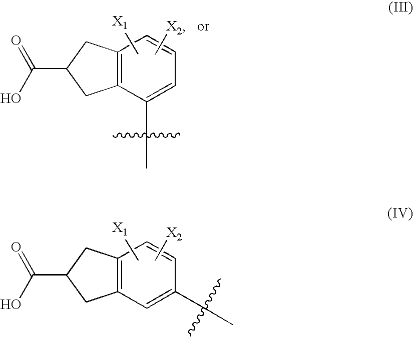
wherein X¹ and X² are each independently selected from the group consisting of hydrogen, halogen, hydroxy, optionally substituted lower alkyl, optionally substituted cycloalkyl, optionally substituted heteroalkyl, optionally substituted cycloheteroalkyl, optionally substituted lower alkynyl, perhaloalkyl, perhaloalkoxy, optionally substituted lower alkoxy, nitro, cyano, and NH₂.

18. The compound as recited in claim 17 wherein:
W is O;
m is 0;
X₁ and X₂ and are each hydrogen; and
G₃ is optionally substituted aryl.

19. The compound as recited in claim 17 wherein:
W is O;
m is 0;
X₁ and X₂ are each hydrogen; and
G³ is optionally substituted heteroaryl.

20. The compound as recited in claim 17 wherein:
W is O;
m is 0;
X₁ and X₂ are each hydrogen; and
G³ is —N═C(R⁷R⁸).

21. The compound as recited in claim 20 wherein at least one of R⁷ and R⁸ is optionally substituted aryl.

22. The compound as recited in claim 21 wherein both R⁷ and R⁸ are optionally substituted aryl.

23. The compound as recited in claim 17 wherein:
W is N:
m is 1;
X₁ and X₂ are each hydrogen; and
G³ is optionally substituted aryl.

24. The compound as recited in claim 16 having a structural Formula (V): 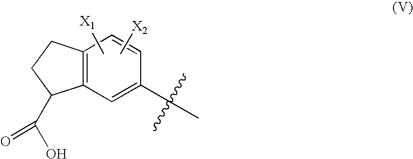
wherein X¹ and X² are each independently selected from the group consisting of hydrogen, halogen, hydroxy, optionally substituted lower alkyl, optionally substituted cycloalkyl, optionally substituted heteroalkyl, optionally substituted cycloheteroalkyl, optionally substituted lower alkynyl, perhaloalkyl, perhaloalkoxy, optionally substituted lower alkoxy, nitro, cyano, and NH₂.

25. The compound as recited in claim 24 wherein:
W is O;
m is 0:
X₁ and X₂ are each hydrogen; and
G₃ is optionally substituted aryl.

26. The compound as recited in claim 1, wherein said compound is selected from the group consisting of Examples 1-20.

27. The compound as recited in claim 1 for use as a medicament.

28. The compound as recited in claim 1 for use in the manufacture of a medicament for the prevention or treatment of a disease or condition ameliorated by the modulation of PPAR-delta.

29. A pharmaceutical composition comprising a compound as recited in claim 1, together with a pharmaceutically acceptable carrier.

30. A method for achieving an effect in a patient comprising the administration of a therapeutically effective amount of a therapeutically effective amount of a compound of Formula I; 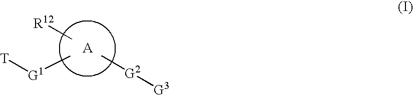
wherein:
A is selected from the group consisting of aryl, heteroaryl, cycloalkyl, and heterocycloalkyl, any of which may be optionally substituted;
R¹² is selected from the group consisting of hydrogen, lower alkyl, lower alkenyl, lower heteroalkyl, and lower alkoxy; R¹² may join together with a carbon atom in G¹ to form a five to eight-membered carbocycle or heterocycle, having structural Formula (II): 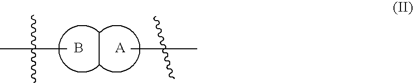
B is a saturated, partially saturated, or unsaturated hydrocarbon chain, optionally containing one or more heteroatoms, to form an optionally substituted five- to eight-membered carbocycle or heterocycle;
T is selected from the group consisting of —C(O)OH, —C(O)NH₂, and tetrazole;
G¹ is selected from the group consisting of —(CR¹R²)ₙ—, -Z(CR¹R²)ᵣ—, (CR¹R²)ᵣZ—, —(CR¹R²)ᵣZ(CR¹R²)ₛ—;
Z is O, S, or NR⁶;
n is 1 to 4;
r and s are 0 to 2;
R¹ and R² are each independently selected from the group consisting of hydrogen, halogen, lower alkyl, lower alkoxy, and lower perhaloalkyl, or R¹ and R² together may form a cycloalkyl;
G² is —Y(CR³R⁴)ₚW(CR³R⁴)ₘ—;
Y is S, —SO₂N(R⁵)— or NR⁶;
W is O, S or —NR⁶;
p is 2 to 6:
m is 0, 1 or 2;
R³ and R⁴ are each independently selected from the group consisting of hydrogen, halogen, hydroxy, optionally substituted lower alkyl, optionally substituted lower alkoxy, optionally substituted heteroalkyl, optionally substituted cycloalkyl, lower perhaloalkyl, lower perhaloalkoxy, nitro, cyano, NH₂, and —C(O)OR¹¹, or R³ and R⁴ together may form a cycloalkyl;
R¹¹ is selected from the group consisting of hydrogen and optionally substituted lower alkyl;
R⁵ and R⁶ are each independently selected from the group consisting of hydrogen, optionally substituted lower alkyl, optionally substituted lower alkenyl, optionally substituted lower alkynyl, optionally substituted heteroalkyl, optionally substituted aryl, and optionally substituted heteroaryl;
G³ is selected from the group consisting of hydrogen, optionally substituted aryl, optionally substituted heteroaryl, optionally substituted cycloalkyl, optionally substituted cycloheteroalkyl, and —N═C(R⁷R⁸);
R⁷ and R⁸ are each individually selected from the group consisting of hydrogen, optionally substituted aryl, optionally substituted heteroaryl, optionally substituted cycloalkyl, and optionally substituted cycloheteroalkyl; and
wherein said effect is selected from the group consisting of modulation of PPARδ, upregulation of expression of GLUT4 in adipose tissue, reduction of expression of NPC1L1, raising of HDL, lowering of LDLc, shifting of LDL particle size from small dense to normal LDL, inhibition of cholesterol absorption, reduction of triglycerides, decrease of insulin resistance, lowering of blood pressure, promotion of wound healing, reduction of scarring, end treatment of a PPARδ-mediated disease.

31. The method as recited in claim 30 wherein said PPARδ-mediated disease is selected from the group consisting of obesity, diabetes, hyperinsulinemia, metabolic syndrome X, dyslipidemia, hypercholesterolemia, cardiovascular disease, vascular disease, atherosclerosis, coronary heart disease, cerebrovascular disease, heart failure, peripheral vessel disease, hyperproliferative disorders, cancers, inflammatory diseases, asthma, rheumatoid arthritis, osteoarthritis, disorders associated with oxidative stress, inflammatory response to tissue injury, psoriasis, ulcerative colitis, dermatitis, autoimmune disease, ophthalmologic diseases, dry eye, macular degeneration, closed angle glaucoma, wide angle glaucoma, inflammation of the eye, and pain of the eye.
